(19) United States
(12) Patent Application Publication
(10) Pub. No.: US 2025/0129157 A1
(43) Pub. Date: Apr. 24, 2025

(54) METHODS AND COMPOSITIONS FOR TREATING MULTIPLE SCLEROSIS

(71) Applicants: Zenas BioPharma, Inc., Waltham, MA (US); Xencor, Inc., Pasadena, CA (US)

(72) Inventors: Steven Kerfoot, London, (CA); Mark Matijevic, Waltham, MA (US); David E. Szymkowski, Pasadena, CA (US); Seung Yup Chu, Pasadena, CA (US)

(21) Appl. No.: 18/904,646

(22) Filed: Oct. 2, 2024

Related U.S. Application Data
(60) Provisional application No. 63/587,401, filed on Oct. 2, 2023.
(60) Provisional application No. 63/595,992, filed on Nov. 3, 2023.

(51) Int. Cl. C07K 16/28; A61K 39/00; A61P 37/00

(52) U.S. Cl. CPC C07K 16/2803; A61P 37/00; A61K 2039/505; A61K 2039/54; A61K 2039/545; C07K 2317/76

(57) ABSTRACT

The present invention provides methods of treating and improving the symptoms of multiple sclerosis using an antibody or antigen-binding fragment thereof that specifically binds human CD19 (e.g., obexclimab).

### CROSS REFERENCE TO RELATED APPLICATIONS

The present application claims benefit of U.S. Provisional Patent Application Nos. 63/587,401, filed on Oct. 2, 2023, and 63/595,992, filed on Nov. 3, 2023, both of which are hereby incorporated by reference in their entirety.

### SEQUENCE LISTING

The present application contains a Sequence Listing which has been submitted electronically in XML file format and is hereby incorporated by reference in its entirety. Said XML copy, created Oct. 2, 2024, is named ZEN-017US1_SL and is 13,137 bytes.

### BACKGROUND

Multiple sclerosis (MS) is a chronic autoimmune disease of the central nervous system (CNS). While the exact cause and mechanisms of the disease are unclear, MS is characterized by inflammation, demyelination, and neurodegeneration along with the formation of plaques or lesions within the CNS. Symptoms of MS are often varied and difficult to predict, but individuals with MS may experience pain, fatigue, cognitive difficulties, depression, mobility restrictions, lack of coordination, difficulties with speech or swallowing, and various vision disorders, as well as many other symptoms. While disease-modifying therapies have been approved for the treatment of MS, there is currently no known cure for MS.

Current anti-CD20-based therapies for MS indiscriminately deplete nearly all B cells (Graf et al., 2021; Lee, D. S. W., et al., 2021. Nat. Rev. Drug Discov. 20:179-199; and Margoni, M., et al., 2022. J. Neurol. 269:1316-1334). People living with MS on long-term treatment could conceivably go for decades missing much of a major branch of the immune system that is necessary for effective and protective immune responses. The long-term health outcomes of maintaining B cell depletion for much of a person's lifespan is not known but could leave individuals less able to mount protective responses to vaccines and increase susceptibility to infection. Further, when the choice is made for the individual to come off anti-CD20 therapy, return to normal immune function could potentially take months while B cells repopulate from the bone marrow (Margoni et al., 2022).

### SUMMARY

The present invention provides a safer and more effective treatment of MS based on an anti-CD19 antibody such as obexelimab. Among other things, an anti-CD19 antibody based therapy (e.g., an obexelimab based therapy) described herein could significantly reduce side effects and substantially benefit subjects with MS who, due to a significant risk of immunosuppression (as highlighted by the COVID pandemic) need to end treatment and restore protective immune function. It is contemplated that other anti-CD19 antibodies with the ability to reduce or inhibit B cells without depleting can also be used to treat MS.

In one aspect, the present invention provides a method of treating multiple sclerosis in a subject in need thereof comprising administering to the subject an anti-CD19 antibody comprising a light chain and a heavy chain, wherein the light chain comprises a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4, and wherein the heavy chain comprises a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7, and wherein the heavy chain comprises an Fc region comprising amino acid substitutions 267E and L328F.

In some aspects, the present invention provides a method of treating multiple sclerosis in a subject in need thereof including administering to the subject an anti-CD19 antibody comprising a light chain and a heavy chain, wherein the light chain contains a light chain variable region that is at least 90% identical to an amino acid sequence of SEQ ID NO: 11, and wherein the heavy chain contains a heavy chain variable region that is at least 90% identical to an amino acid sequence of SEQ ID NO: 12, and wherein the heavy chain contains an Fc region comprising amino acid substitutions 267E and L328F. In some embodiments, an anti-CD19 antibody includes a light chain containing a light chain variable region at least 91%, 92%, 93%, 94%, 95%, 96%, 97%, 98% or 99% identical to SEQ ID NO: 11. In some embodiments, an anti-CD19 antibody contains a heavy chain comprising a heavy chain variable region at least 91%, 92%, 93%, 94%, 95%, 96%, 97%, 98% or 99% identical to SEQ ID NO: 12. In some embodiments, the anti-CD19 antibody contains a heavy chain variable region containing an amino acid sequence of SEQ ID NO: 12; and a light chain variable region containing an amino acid sequence of SEQ ID NO: 11.

In some embodiments, an anti-CD19 antibody includes a light chain containing a light chain variable region at least 80%, 85%, 90%, 91%, 92%, 93%, 94%, 95%, 96%, 97%, 98% or 99% identical to SEQ ID NO: 11. In some embodiments, an anti-CD19 antibody includes a heavy chain containing a heavy chain variable region at least 80%, 85%, 90%, 91%, 92%, 93%, 94%, 95%, 96%, 97%, 98% or 99% identical to SEQ ID NO: 12. In some embodiments, an anti-CD19 antibody includes a light chain and a heavy chain, wherein the light chain contains a light chain variable region at least 80%, 85%, 90%, 91%, 92%, 93%, 94%, 95%, 96%, 97%, 98% or 99% identical to SEQ ID NO: 11, and wherein the heavy chain contains a heavy chain variable region at least 80%, 85%, 90%, 91%, 92%, 93%, 94%, 95%, 96%, 97%, 98% or 99% identical to SEQ ID NO: 12. In some embodiments, the anti-CD19 antibody includes a heavy chain and a light chain, wherein the heavy chain contains an amino acid sequence at least 90% identical to SEQ ID NO: 10; and wherein the light chain contains an amino acid sequence at least 90% identical to SEQ ID NO: 9. In some embodiments, the anti-CD19 antibody includes a heavy chain containing an amino acid sequence of SEQ ID NO: 10; and a light chain containing an amino acid sequence of SEQ ID NO: 9.

In some embodiments, the anti-CD19 antibody is obexelimab.

In some embodiments, the administration of the anti-CD19 antibody improves, ameliorates, or delays the onset of one or more symptoms of MS.

In some embodiments, the anti-CD19 antibody is administered intrathecally.

In some embodiments, the anti-CD19 antibody is administered intravenously.

In some embodiments, the anti-CD19 antibody is administered subcutaneously.

In some embodiments, obexelimab is administered in a liquid formulation comprising 125 mg/ml obexelimab, 2.35 mg/ml sodium acetate trihydrate, 0.17 mg/ml acetic acid, 30 mg/ml L-proline, 0.1 mg/ml polysorbate 80 at pH 5.5.

In some embodiments, the anti-CD19 antibody is administered using a prefilled syringe or autoinjector.

### BRIEF DESCRIPTION OF THE DRAWINGS

Drawings are for illustration purposes and not for limitation.

FIG. 1A-1B: illustrates prevention of induction of disease in an experimental autoimmune encephalomyelitis (“EAE”) model. An obexelimab surrogate antibody was evaluated versus a rituximab surrogate antibody and vehicle control in a preclinical EAE model. FIG. 1A shows prevention of disease by the obexelimab surrogate, as measured by standard clinical EAE scores of the surviving mice at each timepoint (the number of surviving mice per treatment group at each timepoint as indicated below the x-axis). FIG. 1B shows that the obexelimab surrogate was not associated with a significant reduction of blood B cells as compared to the decrease measured in mice treated with the rituximab surrogate (p<0.0001).

FIG. 2A-2B: illustrates inhibition of ovalbumin (OVA) antigen (Ag) uptake and Ag presenting cell (APC) function of murine B cells. FIG. 2A shows inhibition of follicular (Fo) B cell uptake of Ag-coated beads by the obexelimab murine surrogate (mObx; 2480E), compared to anti-CD19 idiotype (2480F) as measured by flow cytometry. Internalized vs external beads bound to the cell membrane were identified by secondary stain. Percent of the total Fo B cell subset with internalized beads is shown. Each symbol represents cells isolated from an individual mouse (**p<0.01, ****p<0.0001). FIG. 2B shows T cell activation by Ag-loaded B cells is inhibited by mObx ex vivo. OVA-sp T cells (purified OT-II TCR transgenic T cells) were labelled with Cell Tracker Violet (CTViolet) and cocultured with B cells with no Ag (media) or loaded with anti-IgM biotinylated OVA coated beads (all other conditions), and either mObx or control. Flow cytometry was used to identify proliferating T cells that had diluted intracellular CTViolet.

FIG. 3A-F: illustrates reduction of BCR-mediated antigen uptake by murine Fo B2 (Fo B), marginal zone (MZ) and B1 B cell subsets. Murine B cells were isolated from the spleen and peritoneal cavity of 2B-KIX mice and incubated for 2 hours at 37° C. and 5% CO₂ with 2 μm goat anti-mouse IgM F(ab′) 2 and biotinylated OVA conjugated beads. Cells were pre-treated with mObx (30 μg/ml) (2480E), its idiotype control (30 μg/ml) (rat anti-mouse CD19 with no Fc effect; 2480F) or cytochalasin D (30 μg/ml) for 1 hour at 37° C. and 5% CO₂. FIG. 3A shows a fixable viable dye (FVD) used to differentiate live and dead cells prior to identification of the B cell subsets. FIG. 3B shows Fo B cells were identified using FACS as B220+, CD4−, CD11b−, IgMlo, IgDhi, CD21int, CD23+. FIG. 3C shows MZ B cells identified using FACS as B220+, CD4−, CD11b−, IgMhi, IgDlo, CD21hi, CD23int. FIG. 3D shows B1 cells identified as B220+, CD4−, F4/80−, IgMhi, IgDlo, CD21−, CD23−. FIG. 3E shows a streptavidin stain used to differentiate internalized bead from extracellular beads. FIG. 3F shows mean percentage of Fo B, MZ and B1 cells with internalized goat anti-mouse IgM F(ab′) 2 coated beads. Each symbol represents data from cells isolated from an individual mouse (n=4) (*p<0.05, **p<0.01, ***p<0.001, ****p<0.0001, ns not significant).

FIG. 4A-F: illustrates reduction of BCR-mediated antigen uptake by human Fo B, MZ and B1 B cell subsets. Primary human B cells were isolated from healthy donors and pre-treated with Obx (30 μg/ml) or cytochalasin D (30 μg/ml) for 1 hour at 37° C. and 5% CO₂. Flow cytometry was subsequently used to assess B cell phagocytosis. FIG. 4A shows a fixable viable dye (FVD) used to differentiate live and dead cells prior to identification of the B cell subsets. FIG. 4B shows Fo B cells identified using FACS as CD20+, CD4−, CD27−, CD43−, CD70−. FIG. 4C shows MZ B cells identified using FACS as CD20+, CD4−, CD27+, IgDhi, IgMhi. FIG. 4D shows B1 like cells identified as CD20+, CD4−, CD27+, CD43+, CD70−. FIG. 4E shows a streptavidin stain used to differentiate internalized beads from extracellular beads. FIG. 4F shows mean percentage of Fo B, MZ and B1 like cells with internalized anti-human IgM coated beads. Each symbol represents data from cells isolated from an individual donor (n=4) (*p<0.05, ns not significant).

FIG. 5A-I: illustrates mObx treated B cells displayed a reduced activation phenotype. Murine B cells were isolated from the spleen and lymph nodes of 2B-KIX mice and OT-II mice. 2B-KIX B cells were co-cultured with OT-II T cells and anti-mouse IgM biotinylated OVA conjugated beads for 4 days at 37° C. 5% CO₂. Prior to incubation with beads, some cells were pre-treated with mObx (30 μg/ml), or its idiotype control (30 μg/ml) for 1 hour at 37° C. 5% CO₂. FIG. 5A shows flow cytometry scatter plots of B cells identified as CD45R+CD4−. FIG. 5B shows the histogram of CD80. FIG. 5C shows the mean fluorescence intensity (MFI) of CD80 for the different treatment conditions. FIG. 5D shows the histogram of CD86. FIG. 5E shows the MFI of CD86 for the different treatment conditions. FIG. 5F shows the histogram of CD11b. FIG. 5G shows the MFI of CD11b for the different treatment conditions. FIG. 5H shows the histogram of CD11c. FIG. 5I shows the MFI of CD11e for the different treatment conditions. Each symbol represents data from cells isolated from an individual pair of mice (n=3) (*p<0.05, **p<0.01).

FIG. 6A-G: illustrates that mObx treated B cells have a reduced ability to activate CD4 T cells. Murine B cells were isolated from the spleen and lymph nodes of 2B-KIXmice and OT-II mice. OT-II T cells were stained with CTViolet and co-cultured with 2B-KIX B cells and anti-mouse IgM biotinylated OVA conjugated beads for 4 days at 37° C. 5% CO₂. Prior to incubation with beads, some cells were pre-treated with mObx (30 μg/ml), or its idiotype control (30 μg/ml) for 1 hour at 37° C. 5% CO₂. FIG. 6A shows CD4 T cells identified as CD4⁺ CD45R⁻. FIG. 6B shows representative flow plots showing percentage of CTViolet stained OT-II T cells proliferating. FIG. 6C shows mean percentage of total OT-II T cell proliferation. FIG. 6D shows mean MFI of surface expressed CD44 on OT-II T cells. FIG. 6E shows mean MFI of surface expressed CD25 on CD44⁺ OT-II T cells. FIG. 6F shows representative histograms of MFI of surface expressed CD25 on CD44⁺ OT-II T cells. FIG. 6G shows mean percentage of OT-II CD4 T cells in each proliferation cycle. Each symbol represents data from cells isolated from an individual pair of mice (n=3) (*p<0.05, **p<0.01, ***p<0.001).

### DETAILED DESCRIPTION

The present invention provides, among other things, methods of treating MS in a subject in need thereof comprising administering to the subject an anti-CD19 antibody comprising an Fc region modified by amino acid substitutions S267E and L328F, wherein administration of the anti-CD19 antibody improves, ameliorates or delays onset of one or more symptoms of MS. In some embodiments, the anti-CD19 antibody is obexclimab.

In some embodiments, the one or more symptoms of MS comprise pain, fatigue, cognitive difficulties, depression, mobility restrictions, lack of coordination, difficulties with speech or swallowing, and various vision disorders, as well as many other symptoms.

Various aspects of the invention are described in detail in the following sections. The use of sections is not meant to limit the invention. Each section can apply to any aspect of the invention. In this application, the use of “or” means “and/or” unless stated otherwise.

### Definitions

Described herein are several definitions. Such definitions are meant to encompass grammatical equivalents.

Antibody: The term “antibody” herein is meant to include a protein consisting of one or more polypeptides substantially encoded by all or part of the recognized immunoglobulin genes. The recognized immunoglobulin genes, for example in humans, include the kappa (κ), lambda (1), and heavy chain genetic loci, which together comprise the myriad variable region genes, and the constant region gene gamma (γ) which encodes the IgG (IgG1, IgG2, IgG3, and IgG4) isotype. Antibody herein is meant to include full length antibodies and antibody fragments, and may refer to a natural antibody from any organism, an engineered antibody, or an antibody generated recombinantly for experimental, therapeutic, or other purposes.

Effector Function: The term “effector function” as used herein is meant a biochemical event that results from the interaction of an antibody Fc region with an Fc receptor or ligand. Effector functions include FcγR-mediated effector functions such as ADCC and ADCP, and complement-mediated effector functions such as CDC. Further, effector functions include FcγRIIb-mediated effector functions, such as inhibitory functions, e.g., downregulating, reducing, inhibiting etc., B cell responses (e.g., a humoral immune response).

Fc or Fc region: The terms “Fc” or “Fc region,” as used herein is meant the polypeptide comprising the constant region of an antibody excluding the first constant region immunoglobulin domain and in some cases, part of the hinge. Thus, Fc may refer to the last two constant region immunoglobulin domains of IgG, and the flexible hinge N-terminal to these domains. For IgG, Fc comprises immunoglobulin domains Cgamma2 and Cgamma3 (Cy2 and Cy3) and the hinge between Cgamma1 (Cy1) and Cgamma2. Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V1 58 and F158) and FcγRIIIb (including allotypes FcγRIIIb-NA1 and FcγRIIIb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

Modification: The term “modification” herein is meant an alteration in the physical, chemical, or sequence properties of a protein, polypeptide, antibody, or immunoglobulin. Modifications described herein include amino acid modifications (including amino acid substitutions) and glycoform modifications.

Target Antigen: The term “target antigen” as used herein is meant the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for obexelimab is CD19.

Target cell: The term “target cell” as used herein is meant a cell that expresses a target antigen.

Treating: The term “treating” as used herein is meant to encompass improving, ameliorating, or delaying the onset of one or more symptoms of a disease or disorder.

Obexelimab: The term “Obexelimab” as used herein, is an Fc engineered humanized monoclonal antibody (mAb) that binds to the human B-cell restricted surface antigen CD19 and has enhanced Fc binding to Fcγ receptor IIb (FcγRIIb). The molecule is an IgG1 immunoglobulin with a kappa light chain and 2 amino acid substitutions in the constant portion of the heavy chain. Obexelimab is a monoclonal antibody with a projected mass of approximately 147,426 Da based on the amino acid sequence. The heavy and light chains of obexelimab are given by SEQ ID NO: 10, and SEQ ID NO: 9, respectively.

### Multiple Sclerosis (MS)

MS is a chronic and potentially disabling immune mediated disease of the CNS characterized by the progressive destruction of the myelin. MS manifests diverse neurological symptoms such as motor paralysis, sensory impairment, higher brain dysfunction, visual loss, and dysuria due to infiltration of autoreactive lymphocytes (e.g., T cells or B cells) into the brain, the spinal cord, or the optic nerve, causing inflammations targeting perineural myelin proteins. MS patients develop transient and repetitive inflammations at various sites of the CNS. As each inflammation occurs, neurological symptoms are manifested depending on the inflammation site.

Although the disease mechanisms are not fully understood, B cells play a role in MS disease progression. B cell subsets play several roles in immunity, most of which are beneficial. For example, the B cell lineage is best known for making antibodies, a function not performed by B cells themselves but instead by activated cells that have differentiated into plasma cells. High affinity antibodies are central to protective, pathogen-specific immune responses to infection or following vaccination. However, while high-affinity auto-antigen (Ag)-specific antibodies do contribute to pathology in the MS-related disorders NMOSD and MOGSDs (Jain, R. W., et al., 2022. Nat. Rev. Immunol. 22:513-524), antibody production is not the primary B cell pathological contribution to MS. Indeed, while therapeutic anti-CD20 antibodies effectively eliminate B cells, they do not eliminate antibody-producing plasma cells, nor are antibody levels reduced within the therapeutic timeframe following administration of anti-CD20-based drugs (Graft et al., 2021, Lee et al., 2021, and Margoni et al., 2022). Therefore, B cells must have other functions that contribute to the ongoing inflammatory response that underlies disease progression.

One of the primary candidate mechanisms through which B cells may drive CNS autoimmunity is through the presentation of auto-Ag to T cells. This, combined with any cytokines that the B cell may produce at the same time, could directly activate naïve autoimmune T cells in lymphatic tissue and influence their differentiation into effector subsets. The process of Ag presentation requires the Ag presenting cell (APC) to collect and internalize protein Ags prior to processing them for loading on to major histocompatibility complex (MHC). This peptide: MHC combination is then transported to the cell surface and exposed to migrating T cells that sample local APCs for cognate Ag via their specific T cell Receptor (TCR).

While B cells are described as one of the three “professional” APCs (along with dendritic cells and macrophages), they are not well known as activators of T cells. In fact, their APC function is best understood in the context of the high affinity antibody response where B cell activation rather than that of T cells is the primary outcome. These events occur in specialized structures called germinal centers (GC) within secondary lymphoid tissues. In this case, naïve B cells that encounter an Ag that binds to their specific B cell receptor (BCR) internalize it (and anything physically linked to it) and presents it to already activated T follicular helper (Tfh) cells that are specific for the same antigen. These cognate interactions are necessary to fully activate the B cell and are foundational to the mechanisms that select high affinity B cells in the GC and for differentiation into plasma cells to produce antibody (Haberman, A. M., et al., 2019. Immunol. Rev. 288:10-27; Kerfoot, S. M., et al., 2011. Immunity 34:947-960; Jain, R. W., et al., 2018., Cell Rep. 25:3342-3355.c5; Parham, K. A., et al., 2022. J. Immunol. 209:1703-1712).

Other scenarios where B cells may present Ag to T cells resulting in T cell activation are much less well understood. Because B cells selectively acquire Ag via BCR binding, they are known to be able to concentrate low-concentration Ag much more effectively than other APCs, and for this reason have been implicated in the initiation of some autoimmune T cell responses (Rodríguez-Pinto, D. 2005. Cell. Immunol. 238:67-75). However, B cells that are specific for any given antigen are extremely rare, and typically B cells are physically separated from naïve T cells in lymphoid tissues, so it is not clear when or where within the lymphoid microanatomy such naïve T cell: cognate B cell interactions could occur.

While activated T cells readily infiltrate MS demyelinating lesions, B cells are relatively rare within the CNS parenchyma (Jain and Yong, 2022). Instead, considerable numbers of B cells can collect along with T cells in the meninges, often immediately adjacent to a lesion (Reali, C., R. et al., 2020. Brain Pathol. 30:779-793, Bell, L., et al., 2020. Front. Immunol. 10; Choi, S. R., et al., 2012. Brain J. Neurol. 135:2925-2937). These clusters of lymphocytes can sometimes become sufficiently organized to resemble lymphoid tissues with separate T cell zones and B cell follicles, but more often than not they are a disorganized mix of these cells. Because of their association with lesions and because their presence is associated with more severe disease (Jain and Yong, 2022), there has been a great deal of interest in these clusters as potential locations from which B cells could exert pathological function(s). Because they can resemble secondary lymphoid tissues, these structures may sustain the autoimmune anti-myelin response from behind the Blood Brain/Meningeal Barriers (Pipi, E. et al., 2018. Front. Immunol. 9). In this scenario, B cells may act as APCs to present locally-acquired CNS auto-Ag to recently recruited effector T cells, resulting in their reactivation to drive inflammation targeting oligodendrocytes in the local parenchyma. Therefore, B cells may function as APCs to drive the autoimmune responses from multiple locations, including the peripheral lymphatics and the meninges.

### Disease Models for MS

Only humans develop MS, but certain aspects of disease can be modeled in mice and other species in order to study the underlying pathological mechanisms of human disease. Experimental Autoimmune Encephalomyelitis (EAE) is an umbrella term that covers numerous “artificially” generated anti-myelin autoimmune responses that result in immune-mediated demyelination in the CNS.

Exemplary EAE models and Model Ag Systems that are primarily based on the autoantigen Myelin Oligodendrocyte Glycoprotein (MOG) are described in Table 1. Improved disease models are described in this application, particularly in the examples section.

TABLE 1
Mouse Strains and Model Antigens.

| Category | | Description | Reference |
|---|---|---|---|
| Mouse | 2D2 | Transgenic TCR | Bettelli, E., M. Pagany, H. L. Weiner, |
| Strains | | for MOG epitope | C. Linington, R. A. Sobel, and |
| | | (MOG₃₅₋₅₅) | V. K. Kuchroo. 2003. Myelin oligodendrocyte |
| | | | glycoprotein-specific T cell receptor transgenic |
| | | | mice develop spontaneous autoimmune |
| | | | optic neuritis. J. Exp. Med. 197: 1073-1081. |
| | IgH^{MOG} | MOG-specific BCR | Litzenburger, T., R. Fässler, J. Bauer, H. |
| | | knock-in mice | Lassmann, C. Linington, H. Wekerle, and |
| | | | A. Iglesias. 1998. B lymphocytes producing |
| | | | demyelinating autoantibodies: development |
| | | | and function in gene-targeted transgenic |
| | | | mice. J. Exp. Med. 188: 169-180. |
| Protein | mMOG_{tag} | Based on the | Dang, A. K., R. W. Jain, H. C. Craig, and |
| Antigen | | extracellular domain | S. M. Kerfoot. 2015. B cell recognition of |
| | | of mouse myelin | myelin oligodendrocyte glycoprotein |
| | | oligodendrocyte | autoantigen depends on immunization with |
| | | glycoprotein | protein rather than short peptide, while B |
| | | (MOG₁₋₁₂₅) | cell invasion of the CNS in autoimmunity |
| | | Induces EAE that | does not. J. Neuroimmunol. 278C: 73-84. |
| | | involves both T | |
| | | cells and B cells in | |
| | | recognition of the | |
| | | autoantigen | |
| | bMOG_{tag} | Based on mMOG_{tag} | Hawkins, R. F. W., A. Patenaude, A. Dumas, |
| | | with a “humanized” | R. Jain, Y. Tesfagiorgis, S. M. Kerfoot, |
| | | 33-55 residue via | T. Matsui, M. Gunzer, P. E. Poubelle, |
| | | an S42P mutation | C. Larochelle, M. Pelletier, and L. Valliéres. |
| | | Induces EAE that | 2017. ICAM1+ neutrophils promote chronic |
| | | depends on B cells | inflammation via ASPRV1 in B cell-dependent |
| | | (anti-MOG antibodies) | autoimmune encephalomyelitis. JCI Insight 2. |
| | | for its initiation | |
| | nfMOG_{tag} | High Affinity 2D3 | Jain, R. W., K. A. Parham, Y. Tesfagiorgis, |
| | | TCR Ag: Based on | H. C. Craig, E. Romanchik, and S. M. Kerfoot. |
| | | mMog_{tag} with a | 2018. Autoreactive, Low-Affinity T Cells |
| | | neurofilament | Preferentially Drive Differentiation of |
| | | M-derived epitope | Short-Lived Memory B Cells at the Expense of |
| | | upstream Mog₁₋₁₂₅ | Germinal Center Maintenance. Cell Rep. |
| | | Induces EAE | 25: 3342-3355.e5. |
| | | (characteristics | |
| | | currently under | |
| | | investigation) | |
| | OVAMOG_{tag} | Based on mMog_{tag} | |
| | | with an OVA epitope | |
| | | upstream MOG₁₋₁₂₅ | |
| | | Induces EAE | |
| | | (characteristics | |
| | | currently under | |
| | | investigation | |
| | PLP_{ECD} | Based on peptide | Wilhelm, C. R., M. A. Upadhye, K. L. |
| | | extracellular | Eschbacher, N. J. Karandikar, and A. W. |
| | | domains of myelin | Boyden. 2023. Proteolipid Protein-Induced |
| | | proteolipid protein | Mouse Model of Multiple Sclerosis |
| | | Induces EAE that | Requires B Cell-Mediated Antigen |
| | | depends on | Presentation. J. Immunol. Ji2200721. |
| | | Ag-presentation | |
| | | by B cells | |

### Anti-CD19 Antibodies

According to the present invention, an anti-CD19 antibody is used to treat a human patient suffering from MS. In some embodiments, the anti-CD19 antibody has enhanced Fc binding to FcγRIIb. In some embodiments, the anti-CD19 antibody comprises amino acid modifications S267E and L328F, wherein the amino acid numbering is according to the EU index according to Kabat.

Exemplary anti-CD19 antibodies are described herein.

### Obexelimab

In some embodiments, obexelimab is used to treat a human patient suffering from MS. In one aspect, the present invention provides a method of administering obexelimab for the treatment of MS. Obexelimab is a monoclonal antibody specific for CD19 comprising: a light chain comprising a variable region having:

| | a CDR1 comprising |
|---|---|
| (SEQ ID NO: 2) | |
| | RSSKSLQNVNGNTYLY, |
| | a CDR2 comprising |
| (SEQ ID NO: 3) | |
| | RMSNLNS, |
| | and |
| | a CDR3 comprising |
| (SEQ ID NO: 4) | |
| | MQHLEYPIT; |

and
a heavy chain comprising a variable region having

| | a CDR1 comprising |
|---|---|
| (SEQ ID NO: 5) | |
| | SYVMH, |
| | a CDR2 comprising |
| (SEQ ID NO: 6) | |
| | WIGYINPYNDGTKY, |
| | and |
| | a CDR3 comprising |
| (SEQ ID NO: 7) | |
| | GTYYYGTRVFDY, |

wherein the heavy chain comprises amino acid substitutions in the Fc region S267E and L328F as compared to SEQ ID NO: 8:
wherein the numbering is according to the EU index, as in Kabat.

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVELFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, obexclimab comprises: a light chain comprising amino acid sequence:

(SEQ ID NO: 9)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSENRGEC;

and heavy chain comprising an amino acid sequence:

(SEQ ID NO: 10)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVEHEDPEVKENWYVDGVEVHNAKTKPREEQY
NSTYRVVSVLTVLHQDWLNGKEYKCKVSNKAFPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

TABLE 2
Sequence of Heavy chain and Light chain
amino acid sequence of obexelimab

| Heavy Chain | Light Chain |
|---|---|
| EVQLVESGGGLVKPGGSLKLSCAASGYTFT | DIVMTQSPATLSLSPGERATLSCRSSKSLQNVN |
| SYVMHWVRQAPGKGLEWIGYINPYNDGTKY | GNTYLYWFQQKPGQSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIKRTVAAPSVFIFPPSDEQLKS |
| SASTKGPSVFPLAPSSKSTSGGTAALGCLV | GTASVVCLLNNFYPREAKVQWKVDNALQSGNSQ |
| KDYFPEPVTVSWNSGALTSGVHTFPAVLQS | ESVTEQDSKDSTYSLSSTLTLSKADYEKHKVYA |
| SGLYSLSSVVTVPSSSLGTQTYICNVNHKP | CEVTHQGLSSPVTKSENRGEC (SEQ ID |
| SNTKVDKKVEPKSCDKTHTCPPCPAPELLG | NO: 9) |
| GPSVFLFPPKPKDTLMISRTPEVTCVVVDV | |
| EHEDPEVKFNWYVDGVEVHNAKTKPREEQY | |
| NSTYRVVSVLTVLHQDWLNGKEYKCKVSNK | |
| AFPAPIEKTISKAKGQPREPQVYTLPPSRE | |
| EMTKNQVSLTCLVKGFYPSDIAVEWESNGQ | |
| PENNYKTTPPVLDSDGSFFLYSKLTVDKSR | |
| WQQGNVFSCSVMHEALHNHYTQKSLSLSPG | |
| K (SEQ ID NO: 10) | |
| Heavy Chain | Light Chain |
| Variable Region (VH) | Variable Region (VL) |
| EVQLVESGGGLVKPGGSLKLSCAASGYTFT | DIVMTQSPATLSLSPGERATLSCRSSKSLONVN |
| SYVMHWVRQAPGKGLEWIGYINPYNDGTKY | GNTYLYWFQQKPGQSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIK (SEQ ID NO: 11) |
| S (SEQ ID NO: 12 | |

TABLE 3
Kabat heavy chain and light chain
CDRs of obexelimab.

| CDR | Amino Acid Sequence |
|---|---|
| LCDR1 | RSSKSLQNVNGNTYLY (SEQ ID NO: 2) |
| LCDR2 | RMSNLNS (SEQ ID NO: 3) |
| LCDR3 | MQHLEYPIT (SEQ ID NO: 3) |
| HCDR1 | SYVMH (SEQ ID NO: 5) |
| HCDR2 | WIGYINPYNDGTKY (SEQ ID NO: 6) |
| HCDR3 | GTYYYGTRVFDY (SEQ ID NO: 7) |

In some embodiments, obexelimab comprises a light chain variable region and a heavy chain variable region as given by Table 2. In some embodiments, obexelimab comprises CDRs as given by Table 3.

In some embodiments, an anti-CD19 antibody comprises a light chain comprising an amino acid sequence of SEQ ID NO: 9. In some embodiments, an anti-CD19 antibody comprises a heavy chain comprising an amino acid sequence of SEQ ID NO: 10. In some embodiments, an anti-CD19 antibody comprises a light chain and a heavy chain, wherein the light chain comprises an amino acid sequence of SEQ ID NO: 9, and wherein the heavy chain comprises an amino acid sequence of SEQ ID NO: 10.

In some embodiments, an anti-CD19 antibody comprises a light chain, wherein the light chain comprises a light chain variable region comprising an amino acid sequence of SEQ ID NO: 11. In some embodiments, an anti-CD19 antibody comprises a heavy chain, wherein the heavy chain comprises a heavy chain variable region comprising an amino acid sequence of SEQ ID NO: 12. In some embodiments, an anti-CD19 antibody comprises a light chain and a heavy chain, wherein the light chain comprises a light chain variable region comprising an amino acid sequence of SEQ ID NO: 11, and wherein the heavy chain comprises a heavy chain variable region comprising an amino acid sequence of SEQ ID NO: 10. In some embodiments, an anti-CD19 antibody comprises a heavy chain, wherein the heavy chain comprises a heavy chain variable region comprising an amino acid sequence of SEQ ID NO: 12, and wherein the heavy chain comprises an Fc region comprising amino acid substitutions S267E and L328F. In some embodiments, an anti-CD19 antibody comprises a light chain and a heavy chain, wherein the light chain comprises a light chain variable region comprising an amino acid sequence of SEQ ID NO: 11, and wherein the heavy chain comprises a heavy chain variable region comprising an amino acid sequence of SEQ ID NO: 10, and wherein the heavy chain comprises an Fc region comprising amino acid substitutions S267E and L328F.

In some embodiments, an anti-CD19 antibody comprises a light chain comprising a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4. In some embodiments, an anti-CD19 antibody comprises a heavy chain comprising a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7. In some embodiments, an anti-CD19 antibody comprises a heavy chain comprising a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7, and wherein the heavy chain comprises an Fc region comprising amino acid substitutions S267E and L328F. In some embodiments, an anti-CD19 antibody comprises a light chain and heavy chain, wherein the light chain comprises a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4, and wherein the heavy chain comprises a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7. In some embodiments, an anti-CD19 antibody comprises a light chain and heavy chain, wherein the light chain comprises a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4, and wherein the heavy chain comprises a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7, and wherein the heavy chain comprises an Fc region comprising amino acid substitutions S267E and L328F.

Obexelimab works by exploiting the regulation of B-cell receptor (BCR) signaling by FcγRIIb. Obexelimab binds CD19 of the BCR complex and its Fc is engineered to increase its affinity for the inhibitory FcγRIIb. Since CD19 is associated with the BCR, Obexelimab tethering of CD19 to FcγRIIb on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it may not mediate B cell killing.

### Variants

In some embodiments, a variant of obexelimab is an immunoglobulin specific for CD19 comprises: a light chain comprising a variable region having a CDR1 comprising RSSKSLONVNGNTYLY, a CDR2 comprising RMSNLNS, and a CDR3 comprising MQHLEYPIT; and a heavy chain comprising a variable region having a CDR1 comprising SYVMH, a CDR2 comprising WIGYINPYNDGTKY, and a CDR3 comprising GTYYYGTRVFDY, wherein the heavy chain comprises amino acid substitutions in the Fc region S267E and L328F as compared to:

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVELFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

wherein the numbering is according to the EU index, as in Kabat.

In some embodiments, a variant of obexelimab comprises a heavy chain variable region (VH) and/or a light chain variable region (VL), comprising a CDR1, a CDR2, and a CDR3, each of which differs by no more than 1, 2, 3, 4 or 5 amino acid residues from each of RSSKSLQNVNGNTYLY (SEQ ID NO: 2), RMSNLNS (SEQ ID NO: 3), MQHLEYPIT (SEQ ID NO: 4), SYVMH (SEQ ID NO: 5), WIGYINPYNDGTKY (SEQ ID NO: 6) and/or GTYYYGTRVFDY (SEQ ID NO: 7). In some embodiments, a variant of obexclimab comprises a heavy chain, wherein the heavy chain comprises an Fc region comprising amino acid substitutions S267E and L328F.

In some embodiments, an anti-CD19 antibody comprises a light chain comprising a LCDR1, LCDR2, and LCDR3, each of which differs by no more than 1 residue from each of SEQ ID NO: 2, SEQ ID NO: 3, and SEQ ID NO: 4. In some embodiments, an anti-CD19 antibody comprises a light chain comprising a LCDR1, LCDR2, and LCDR3, each of which differs by no more than 2 residues from each of SEQ ID NO: 2, SEQ ID NO: 3, and SEQ ID NO: 4. In some embodiments, an anti-CD19 antibody comprises a light chain comprising a LCDR1, LCDR2, and LCDR3, each of which differs by no more than 3 residues from each of SEQ ID NO: 2, SEQ ID NO: 3, and SEQ ID NO: 4. In some embodiments, an anti-CD19 antibody comprises a light chain comprising a LCDR1, LCDR2, and LCDR3, each of which differs by no more than 4 residue from each of SEQ ID NO: 2, SEQ ID NO: 3, and SEQ ID NO: 4. In some embodiments, an anti-CD19 antibody comprises a light chain comprising a LCDR1, LCDR2, and LCDR3, each of which differs by no more than 5 residues from each of SEQ ID NO: 2, SEQ ID NO: 3, and SEQ ID NO: 4.

In some embodiments, an anti-CD19 antibody comprises a heavy chain comprising a HCDR1, HCDR2, and HCDR3, each of which differs by no more than 1 residue from each of SEQ ID NO: 5, SEQ ID NO: 6, and SEQ ID NO: 7. In some embodiments, an anti-CD19 antibody comprises a heavy chain comprising a HCDR1, HCDR2, and HCDR3, each of which differs by no more than 2 residues from each of SEQ ID NO: 5, SEQ ID NO: 6, and SEQ ID NO: 7. In some embodiments, an anti-CD19 antibody comprises a heavy chain comprising a HCDR1, HCDR2, and HCDR3, each of which differs by no more than 3 residues from each of SEQ ID NO: 5, SEQ ID NO: 6, and SEQ ID NO: 7. In some embodiments, an anti-CD19 antibody comprises a heavy chain comprising a HCDR1, HCDR2, and HCDR3, each of which differs by no more than 4 residues from each of SEQ ID NO: 5, SEQ ID NO: 6, and SEQ ID NO: 7. In some embodiments, an anti-CD19 antibody comprises a heavy chain comprising a HCDR1, HCDR2, and HCDR3, each of which differs by no more than 5 residues from each of SEQ ID NO: 5, SEQ ID NO: 6, and SEQ ID NO: 7.

In some embodiments, an anti-CD19 antibody comprises a heavy chain comprising a HCDR1, HCDR2, and HCDR3, each of which differs by no more than 1 residue from each of SEQ ID NO: 5, SEQ ID NO: 6, and SEQ ID NO: 7, and wherein the heavy chain comprises an Fc region comprising amino acid substitutions S267E and L328F. In some embodiments, an anti-CD19 antibody comprises a heavy chain comprising a HCDR1, HCDR2, and HCDR3, each of which differs by no more than 2 residues from each of SEQ ID NO: 5, SEQ ID NO: 6, and SEQ ID NO: 7, and wherein the heavy chain comprises an Fc region comprising amino acid substitutions S267E and L328F. In some embodiments, an anti-CD19 antibody comprises a heavy chain comprising a HCDR1, HCDR2, and HCDR3, each of which differs by no more than 3 residues from each of SEQ ID NO: 5, SEQ ID NO: 6, and SEQ ID NO: 7, and wherein the heavy chain comprises an Fc region comprising amino acid substitutions S267E and L328F. In some embodiments, an anti-CD19 antibody comprises a heavy chain comprising a HCDR1, HCDR2, and HCDR3, each of which differs by no more than 4 residues from each of SEQ ID NO: 5, SEQ ID NO: 6, and SEQ ID NO: 7, and wherein the heavy chain comprises an Fc region comprising amino acid substitutions S267E and L328F. In some embodiments, an anti-CD19 antibody comprises a heavy chain comprising a HCDR1, HCDR2, and HCDR3, each of which differs by no more than 5 residues from each of SEQ ID NO: 5, SEQ ID NO: 6, and SEQ ID NO: 7, and wherein the heavy chain comprises an Fc region comprising amino acid substitutions S267E and L328F.

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 2. In some embodiments, the variant of obexelimab comprises: a heavy chain variable region comprising an amino acid sequence that is at least 90%, 91%, 92%, 93%, 94%, 95%, 96%, 97%, 98% or 99% identical to the heavy chain variable region as identified in Table 2.

In some embodiments, an anti-CD19 antibody comprises a light chain comprising a light chain variable region at least 80% identical to SEQ ID NO: 11. In some embodiments, an anti-CD19 antibody comprises a light chain comprising a light chain variable region at least 85% identical to SEQ ID NO: 11. In some embodiments, an anti-CD19 antibody comprises a light chain comprising a light chain variable region at least 90% identical to SEQ ID NO: 11. In some embodiments, an anti-CD19 antibody comprises a light chain comprising a light chain variable region at least 91% identical to SEQ ID NO: 11. In some embodiments, an anti-CD19 antibody comprises a light chain comprising a light chain variable region at least 92% identical to SEQ ID NO: 11. In some embodiments, an anti-CD19 antibody comprises a light chain comprising a light chain variable region at least 93% identical to SEQ ID NO: 11. In some embodiments, an anti-CD19 antibody comprises a light chain comprising a light chain variable region at least 94% identical to SEQ ID NO: 11. In some embodiments, an anti-CD19 antibody comprises a light chain comprising a light chain variable region at least 95% identical to SEQ ID NO: 11. In some embodiments, an anti-CD19 antibody comprises a light chain comprising a light chain variable region at least 96% identical to SEQ ID NO: 11. In some embodiments, an anti-CD19 antibody comprises a light chain comprising a light chain variable region at least 97% identical to SEQ ID NO: 11. In some embodiments, an anti-CD19 antibody comprises a light chain comprising a light chain variable region at least 98% identical to SEQ ID NO: 11. In some embodiments, an anti-CD19 antibody comprises a light chain comprising a light chain variable region at least 99% identical to SEQ ID NO: 11.

In some embodiments, an anti-CD19 antibody comprises a heavy chain comprising a heavy chain variable region at least 80% identical to SEQ ID NO: 12. In some embodiments, an anti-CD19 antibody comprises a heavy chain comprising a heavy chain variable region at least 85% identical to SEQ ID NO: 12. In some embodiments, an anti-CD19 antibody comprises a heavy chain comprising a heavy chain variable region at least 90% identical to SEQ ID NO: 12. In some embodiments, an anti-CD19 antibody comprises a heavy chain comprising a heavy chain variable region at least 91% identical to SEQ ID NO: 12. In some embodiments, an anti-CD19 antibody comprises a heavy chain comprising a heavy chain variable region at least 92% identical to SEQ ID NO: 12. In some embodiments, an anti-CD19 antibody comprises a heavy chain comprising a heavy chain variable region at least 93% identical to SEQ ID NO: 12. In some embodiments, an anti-CD19 antibody comprises a heavy chain comprising a heavy chain variable region at least 94% identical to SEQ ID NO: 12. In some embodiments, an anti-CD19 antibody comprises a heavy chain comprising a heavy chain variable region at least 95% identical to SEQ ID NO: 12. In some embodiments, an anti-CD19 antibody comprises a heavy chain comprising a heavy chain variable region at least 96% identical to SEQ ID NO: 12. In some embodiments, an anti-CD19 antibody comprises a heavy chain comprising a heavy chain variable region at least 97% identical to SEQ ID NO: 12. In some embodiments, an anti-CD19 antibody comprises a heavy chain comprising a heavy chain variable region at least 98% identical to SEQ ID NO: 12. In some embodiments, an anti-CD19 antibody comprises a heavy chain comprising a heavy chain variable region at least 99% identical to SEQ ID NO: 12.

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 2 and a heavy chain variable region comprising an amino acid sequence that is at least 90%, 91%, 92%, 93%, 94%, 95%, 96%, 97%, 98% or 99% identical to the heavy chain variable region as identified in Table 2 and further comprises amino acid substitutions in the Fc region S267E and L328F as compared to SEQ ID NO: 8, wherein the numbering is according to the EU index.

In some embodiments, an anti-CD19 antibody comprises a light chain and a heavy chain, wherein the light chain comprises a light chain variable region at least 80% identical to SEQ ID NO: 11, and wherein the heavy chain comprises a heavy chain variable region at least 80% identical to SEQ ID NO: 12. In some embodiments, an anti-CD19 antibody comprises a light chain and a heavy chain, wherein the light chain comprises a light chain variable region at least 85% identical to SEQ ID NO: 11, and wherein the heavy chain comprises a heavy chain variable region at least 85% identical to SEQ ID NO: 12. In some embodiments, an anti-CD19 antibody comprises a light chain and a heavy chain, wherein the light chain comprises a light chain variable region at least 90% identical to SEQ ID NO: 11, and wherein the heavy chain comprises a heavy chain variable region at least 90% identical to SEQ ID NO: 12. In some embodiments, an anti-CD19 antibody comprises a light chain and a heavy chain, wherein the light chain comprises a light chain variable region at least 91% identical to SEQ ID NO: 11, and wherein the heavy chain comprises a heavy chain variable region at least 91% identical to SEQ ID NO: 12. In some embodiments, an anti-CD19 antibody comprises a light chain and a heavy chain, wherein the light chain comprises a light chain variable region at least 92% identical to SEQ ID NO: 11, and wherein the heavy chain comprises a heavy chain variable region at least 92% identical to SEQ ID NO: 12. In some embodiments, an anti-CD19 antibody comprises a light chain and a heavy chain, wherein the light chain comprises a light chain variable region at least 93% identical to SEQ ID NO: 11, and wherein the heavy chain comprises a heavy chain variable region at least 93% identical to SEQ ID NO: 12. In some embodiments, an anti-CD19 antibody comprises a light chain and a heavy chain, wherein the light chain comprises a light chain variable region at least 94% identical to SEQ ID NO: 11, and wherein the heavy chain comprises a heavy chain variable region at least 94% identical to SEQ ID NO: 12. In some embodiments, an anti-CD19 antibody comprises a light chain and a heavy chain, wherein the light chain comprises a light chain variable region at least 95% identical to SEQ ID NO: 11, and wherein the heavy chain comprises a heavy chain variable region at least 95% identical to SEQ ID NO: 12. In some embodiments, an anti-CD19 antibody comprises a light chain and a heavy chain, wherein the light chain comprises a light chain variable region at least 96% identical to SEQ ID NO: 11, and wherein the heavy chain comprises a heavy chain variable region at least 96% identical to SEQ ID NO: 12. In some embodiments, an anti-CD19 antibody comprises a light chain and a heavy chain, wherein the light chain comprises a light chain variable region at least 97% identical to SEQ ID NO: 11, and wherein the heavy chain comprises a heavy chain variable region at least 97% identical to SEQ ID NO: 12. In some embodiments, an anti-CD19 antibody comprises a light chain and a heavy chain, wherein the light chain comprises a light chain variable region at least 98% identical to SEQ ID NO: 11, and wherein the heavy chain comprises a heavy chain variable region at least 98% identical to SEQ ID NO: 12. In some embodiments, an anti-CD19 antibody comprises a light chain and a heavy chain, wherein the light chain comprises a light chain variable region at least 99% identical to SEQ ID NO: 11, and wherein the heavy chain comprises a heavy chain variable region at least 99% identical to SEQ ID NO: 12.

In some embodiments, the variant of obexelimab comprises: a light chain comprising an amino acid sequence 70%, 75%, 80%, 85%, 90%, 95%, 98% or 99% identical to the light chain as identified in Table 2. In some embodiments, the variant of obexelimab comprises: a heavy chain comprising an amino acid sequence 70%, 75%, 80%, 85%, 90%, 95%, 98% or 99% identical to the heavy chain as identified in Table 2.

In some embodiments, an anti-CD19 antibody comprises a light chain amino acid sequence that is at least 70% identical to SEQ ID NO: 9. In some embodiments, an anti-CD19 antibody comprises a light chain amino acid sequence that is at least 75% identical to SEQ ID NO: 9. In some embodiments, an anti-CD19 antibody comprises a light chain amino acid sequence that is at least 80% identical to SEQ ID NO: 9. In some embodiments, an anti-CD19 antibody comprises a light chain amino acid sequence that is at least 85% identical to SEQ ID NO: 9. In some embodiments, an anti-CD19 antibody comprises a light chain amino acid sequence that is at least 90% identical to SEQ ID NO: 9. In some embodiments, an anti-CD19 antibody comprises a light chain amino acid sequence that is at least 91% identical to SEQ ID NO: 9. In some embodiments, an anti-CD19 antibody comprises a light chain amino acid sequence that is at least 92% identical to SEQ ID NO: 9. In some embodiments, an anti-CD19 antibody comprises a light chain amino acid sequence that is at least 93% identical to SEQ ID NO: 9. In some embodiments, an anti-CD19 antibody comprises a light chain amino acid sequence that is at least 94% identical to SEQ ID NO: 9. In some embodiments, an anti-CD19 antibody comprises a light chain amino acid sequence that is at least 95% identical to SEQ ID NO: 9. In some embodiments, an anti-CD19 antibody comprises a light chain amino acid sequence that is at least 96% identical to SEQ ID NO: 9. In some embodiments, an anti-CD19 antibody comprises a light chain amino acid sequence that is at least 97% identical to SEQ ID NO: 9. In some embodiments, an anti-CD19 antibody comprises a light chain amino acid sequence that is at least 98% identical to SEQ ID NO: 9. In some embodiments, an anti-CD19 antibody comprises a light chain amino acid sequence that is at least 99% identical to SEQ ID NO: 9.

In some embodiments, an anti-CD19 antibody comprises a heavy chain amino acid sequence that is at least 70% identical to SEQ ID NO: 10. In some embodiments, an anti-CD19 antibody comprises a heavy chain amino acid sequence that is at least 75% identical to SEQ ID NO: 10. In some embodiments, an anti-CD19 antibody comprises a heavy chain amino acid sequence that is at least 80% identical to SEQ ID NO: 10. In some embodiments, an anti-CD19 antibody comprises a heavy chain amino acid sequence that is at least 85% identical to SEQ ID NO: 10. In some embodiments, an anti-CD19 antibody comprises a heavy chain amino acid sequence that is at least 90% identical to SEQ ID NO: 10. In some embodiments, an anti-CD19 antibody comprises a heavy chain amino acid sequence that is at least 91% identical to SEQ ID NO: 10. In some embodiments, an anti-CD19 antibody comprises a heavy chain amino acid sequence that is at least 92% identical to SEQ ID NO: 10. In some embodiments, an anti-CD19 antibody comprises a heavy chain amino acid sequence that is at least 93% identical to SEQ ID NO: 10. In some embodiments, an anti-CD19 antibody comprises a heavy chain amino acid sequence that is at least 94% identical to SEQ ID NO: 10. In some embodiments, an anti-CD19 antibody comprises a heavy chain amino acid sequence that is at least 95% identical to SEQ ID NO: 10. In some embodiments, an anti-CD19 antibody comprises a heavy chain amino acid sequence that is at least 96% identical to SEQ ID NO: 10. In some embodiments, an anti-CD19 antibody comprises a heavy chain amino acid sequence that is at least 97% identical to SEQ ID NO: 10. In some embodiments, an anti-CD19 antibody comprises a heavy chain amino acid sequence that is at least 98% identical to SEQ ID NO: 10. In some embodiments, an anti-CD19 antibody comprises a heavy chain amino acid sequence that is at least 99% identical to SEQ ID NO: 10.

In some embodiments, the variant of obexelimab comprises: a light chain comprising an amino acid sequence that is at least 90%, 91%, 92%, 93%, 94%, 95%, 96%, 97%, 98% or 99% identical to the light chain sequence as identified in Table 2 and a heavy chain comprising an amino acid sequence that is at least 90%, 91%, 92%, 93%, 94%, 95%, 96%, 97%, 98% or 99% identical to the heavy chain sequence as identified in Table 2, and the heavy chain of the variant comprises amino acid substitutions in the Fc region S267E and L328F as compared to SEQ ID NO: 8, wherein the numbering is according to the EU index, as in Kabat.

In some embodiments, a suitable variant of obexelimab binds to the same epitope on human CD19, as an antibody comprising a light chain and a heavy chain as identified in Table 2. Epitope binding may be determined by a method known in the art.

In some embodiments, a suitable variant of obexelimab competes for binding to human CD19, as an antibody comprising a light chain and heavy chain as identified in Table 2, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, monoclonal antibody candidates are screened pairwise for binding to a target protein. Other standard methods involve ELISA-based screens and may require synthesis of sets of overlapping peptides corresponding to the protein of interest.

In some embodiments, the human CD19 comprises an amino acid sequence of SEQ ID NO: 1. In some embodiments, an anti-CD19 antibody binds to the extracellular domain of human CD19.

(SEQ ID NO: 1)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

### Fc Receptor Binding Properties

Anti-CD19 antibodies (e.g., obexelimab) disclosed herein comprise an Fc variant that has enhanced Fc binding to the inhibitory Fcγ receptor IIb (FcγRIIb). FcγRIIb, the only FcR on B cells, serves as an antibody-sensing down-regulator of humoral immunity that is naturally engaged by immune complexes. When sufficient antibody is raised against a given antigen, specific immune complexes form and co-engage FcγRIIb and the B cell receptor (BCR) with high avidity, selectively suppressing only B cells recognizing cognate antigen. In addition, FcγRIIb regulates the activity of other B cell stimulators including interleukin (IL)-4, LPS, and BAFF that amplify BCR-driven proliferation and differentiation. By simultaneously binding CD19 and FcγRIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIIb. In other embodiments, immunoglobulins disclosed herein provide enhanced affinity for FcγRIIb, yet reduced affinity for one or more activating FcγRs, including for example FcγRI, FcγRIIa, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIIb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from about two to fifty amino acid modifications, e.g., from about two to ten amino acid modifications, from about two to about five amino acid modifications, etc. compared to the parent. Thus, the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5th Ed., United States Public Health Service, National Institutes of Health, Bethesda, hereby entirely incorporated by reference). In brief, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, Con C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) lgG1 binds FcγRIIb with an affinity of about 1.5 mM, or about 1500 nM. Furthermore, some Fc variants described herein bind FcγRIIb with an affinity about 10-fold greater to WT lgG1. As disclosed herein, greater or enhanced affinity means having a KD lower than about 100 nM, for example between about 10 nM-about 100 nM, between about 1-about 100 nM, or less than about 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136).

An important parameter that determines the most beneficial selectivity of a given Fc variant to treat a given disease is the context of the Fc variant. Thus, the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Treatment of Multiple Sclerosis Using Anti-CD19 Antibodies

In some aspects, the present invention provides a method of treating MS in a subject in need thereof using an anti-CD19 antibody or antigen binding fragment thereof.

In some embodiments, a method of treating MS in a subject in need thereof comprises administering to the subject an anti-CD19 antibody comprising a light chain and a heavy chain, wherein the light chain comprises a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4, and wherein the heavy chain comprises a HCDR1 comprising the an amino acid sequence of SEQ ID NO: 5, a HCDR2 comprising an amino acid sequence of SEQ ID NO: 6, and a HCDR3 comprising an amino acid sequence of SEQ ID NO: 7, and wherein the heavy chain comprises an Fc region comprising amino acid substitutions 267E and L328F.

In some aspects, a method of treating MS in a subject in need thereof comprises administering to the subject an anti-CD19 antibody comprising a light chain and a heavy chain, wherein the light chain comprises a light chain variable region that is at least 90% identical to an amino acid sequence of SEQ ID NO: 11, and wherein the heavy chain comprises a heavy chain variable region comprising an amino acid sequence that is at least 90% identical to an amino acid sequence of SEQ ID NO: 12, and wherein the heavy chain comprises an Fc region comprising amino acid substitutions 267E and L328F.

In some embodiments, a method of treating MS comprises administering obexelimab.

The mode of administration may vary. Suitable routes of administration include various methods known in the art. In some embodiments, an anti-CD19 antibody is administered via injection. In some embodiments, an anti-CD19 antibody is administer intrathecally. In some embodiments, an anti-CD19 antibody is administered intravenously. In some embodiments, an anti-CD19 antibody is administered subcutaneously. In some embodiments, an anti-CD19 antibody is administered using a prefilled syringe or autoinjector.

In some embodiments, administration of the anti-CD19 antibody improves, ameliorates, or delays the onset of one or more symptoms of MS. In some embodiments, administration of the anti-CD19 antibody improves or ameliorates one or more symptoms of MS. In some embodiments, administration of the anti-CD19 antibody delays the onset of one or more symptoms of MS.

### Formulation and Pharmaceutical Compositions

The present invention provides pharmaceutical compositions and formulations of anti-CD19 antibodies (e.g., obexelimab). The pharmaceutical compositions, formulations and related methods of the invention are useful for delivering anti-CD19 antibodies (e.g., obexelimab) and for the treatment of the MS and associated diseases. Formulations of the anti-CD19 antibody disclosed herein are prepared for storage by mixing said antibody having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions.

In some embodiments, pharmaceutical compositions of interest comprise an anti-CD19 antibody (e.g., obexelimab) at various concentrations. In some embodiments, suitable formulations may comprise the antibody of interest at a concentration up to about 250 mg/ml (e.g., up to about 225 mg/ml, up to 200 mg/ml, up to 150 mg/ml, up to 140 mg/ml, up to 130 mg/ml, up to 125 mg/ml, up to 120 mg/ml, up to 115 mg/ml, up to 110 mg/ml, up to 105 mg/ml, up to 100 mg/ml, up to 90 mg/ml, up to 80 mg/ml, up to 70 mg/ml, up to 60 mg/ml, up to 50 mg/ml, up to 40 mg/ml, up to 30 mg/ml, up to 25 mg/ml, up to 20 mg/ml, up to 10 mg/ml).

In some embodiments, suitable formulations may contain the anti-CD19 antibody at a concentration ranging between about 10-300 mg/ml (e.g., about 10-250 mg/ml, about 10-200 mg/ml, about 10-180 mg/ml, about 10-160 mg/ml, about 10-150 mg/ml, about 10-140 mg/ml, about 10-130 mg/ml, about 10-125 mg/ml, about 100-125 mg/ml, about 100-180 mg/ml, about 100-150 mg/ml, about 100-130 mg/ml, about 100-125 mg/ml, about 100-170 mg/ml, about 100-160 mg/ml, about 100-150 mg/ml, about 100-200 mg/ml, about 120-130 mg/ml).

In some embodiments, formulations suitable for administration may contain an anti-CD19 antibody (e.g., obexelimab) at a concentration of approximately 100 mg/ml, 115 mg/ml, 120 mg/ml, 125 mg/ml, 130 mg/ml, 135 mg/ml, 140 mg/ml, 145 mg/ml, 150 mg/ml, 200 mg/ml or 300 mg/ml.

In some embodiments, isotonic solutions are used. In some embodiments, slightly hypertonic solutions (e.g., up to 300 mM (e.g., up to 250 mM, 200 mM, 175 mM, 150 mM, 125 mM) sodium chloride in 5 mM sodium phosphate at pH 7.0) and sugar-containing solutions (e.g., up to 3% (e.g., up to 2.4%, 2.0%, 1.5%, 1.0%) sucrose in 5 mM sodium phosphate at pH 7.0). In some embodiments, a suitable formulation composition is saline (e.g., 150 mM NaCl in water).

Many therapeutic agents, and in particular the antibodies of the present invention, require controlled pH and specific excipients to maintain their solubility and stability in the pharmaceutical compositions of the present invention.

The pH of the pharmaceutical composition is an additional factor which is capable of altering the solubility of an anti-CD19 antibody (e.g., obexelimab) in an aqueous pharmaceutical composition. In some embodiments, pharmaceutical compositions of the present invention contain one or more buffers. In some embodiments, compositions according to the invention contain an amount of buffer sufficient to maintain the optimal pH of said composition between about 4.0-8.0, between about 5.0-7.5, between about 5.5-7.0, between about 6.0-7.0 and between about 6.0-7.5. In other embodiments, the buffer comprises up to about 50 mM (e.g., up to about 45 mM, 40 mM, 35 mM, 30 mM, 25 mM, 20 mM, 15 mM, 10 mM, 5 mM) of sodium phosphate. Suitable buffers include, for example acetate, succinate, citrate, phosphate, other organic acids and tris (hydroxymethyl) aminomethane (“Tris”).

Suitable buffer concentrations can be from about 1 mM to about 100 mM, or from about 3 mM to about 20 mM, depending, for example, on the buffer and the desired isotonicity of the formulation. In some embodiments, a suitable buffering agent is present at a concentration of approximately 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, formulations contain an isotonicity agent to keep the formulations isotonic. Exemplary isotonicity agents include, but are not limited to, glycine, sorbitol, mannitol, sodium chloride and arginine. In some embodiments, suitable isotonic agents may be present in formulations at a concentration from about 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, formulations may contain a stabilizing agent to protect the antibody. Typically, a suitable stabilizing agent is a non-reducing sugar such as sucrose, raffinose, trehalose, or amino acids such as glycine, arginine and methionine. The amount of stabilizing agent in a formulation is generally such that the formulation will be isotonic. However, hypertonic formulations may also be suitable. In addition, the amount of stabilizing agent must not be too low such that an unacceptable amount of degradation/aggregation of the antibody occurs. Exemplary stabilizing agent concentrations in the formulation may range from about 1 mM to about 400 mM (e.g., from about 30 mM to about 300 mM, and from about 50 mM to about 100 mM), or alternatively, from 0.1% to 15% (e.g., from 1% to 10%, from 5% to 15%, from 5% to 10%) by weight. In some embodiments, the ratio of the mass amount of the stabilizing agent and the therapeutic agent is about 1:1. In other embodiments, the ratio of the mass amount of the stabilizing agent and the therapeutic agent can be about 0.1:1, 0.2:1, 0.25:1, 0.4:1, 0.5:1, 1:1, 2:1, 2.6:1, 3:1, 4:1, 5:1, 10:1, or 20:1. In some embodiments, suitable for lyophilization, the stabilizing agent is also a lyoprotectant.

In some embodiments, it is desirable to add a surfactant to formulations. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g., lauroamidopropyl); myristarnidopropyl-, palmidopropyl-, or isostearamidopropyl-dimethylamine; sodium methyl cocoyl-, or disodium methyl ofcyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from about 0.001-0.5% (e.g., about 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc.

In some embodiments, suitable formulations may further include one or more bulking agents, in particular, for lyophilized formylations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C., 45° C., etc.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

In some embodiments, formulations according to the present invention are in a liquid or aqueous form. In some embodiments, formulations of the present invention are lyophilized. Such lyophilized formulations may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

The pharmaceutical compositions of the present invention are characterized by their tolerability. As used herein, the terms “tolerable” and “tolerability” refer to the ability of the pharmaceutical compositions of the present invention to not elicit an adverse reaction in the patient to whom such composition is administered, or alternatively not to elicit a serious adverse reaction in the patient to whom such composition is administered. In some embodiments, the pharmaceutical compositions of the present invention are well tolerated by the patient to whom such compositions is administered.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibody disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The formulations to be used for in vivo administration may be sterile. In some embodiments, the formulation is sterilized by filtration through sterile filtration membranes.

Nonlimiting examples of buffers include phosphate, citrate, acetate, glutamate, carbonate, tartrate, triethanolamine (TRIS), glycylglycine, histidine, glycine, lysine, arginine, and other organic acids. More specifically, non-limiting examples of buffers include HEPES sodium, MES, potassium phosphate, potassium thiocyanate, sterilant, TAE, TBE, ammonium sulfate/HEPES, BuffAR, sodium acetate, sodium carbonate, sodium citrate, sodium dihydrogen phosphate, disodium hydrogen phosphate, and sodium phosphate. Additionally, the buffer may be various hydrate forms. For example, the buffer may be a monohydrate, a dihydrate, a trihydrate, a tetrahydrate, a pentahydrate, a hexahydrate, a heptahydrate, an octahydrate, a nonahydrate, a decahydrate, an undecahydrate, and a dodecahydrate. Occasionally, a hydrate may be fractional such as a hemihydrate or a sesquihydrate. Nonlimiting examples of tonicity modifies include sodium chloride, acetic acid, L-proline, dextrose, mannitol, potassium chloride, glycerin, and glycerol. Non-limiting example of solvents include water, propylene glycol, polyethylene glycols, ethanol, dimethyl sulfoxide, N-methyl-2-pyrrolidone, glycofurol, Solketal™, glycerol formal, acetone, tetrahydrofurfuryl alcohol, diglyme, dimethyl isosorbide, and ethyl lactate. Non-limiting examples of solvents include polysorbates (e.g., polysorbate-20, polysorbate-80), polyoxyethylene sorbitan monooleate (Tween 80), sorbitan monooleate polyoxyethylene sorbitan monolaurate (Tween 20), sorbitan trioleate (span 85), lecithin, and polyoxyethylene polyoxypropylene copolymers (Pluronics, Pluronic F-68).

The amounts of anti-CD19 antibody (e.g., obexelimab), buffer, tonicity modifier, solvent, and surfactants may vary. In some embodiments, the anti-CD19 antibody (e.g., obexelimab) is formulated at a concentration of 125 mg/ml obexclimab, 2.35 mg/ml sodium acetate trihydrate, 0.17 mg/ml acetic acid (at density 1.053 g/ml), 30 mg/ml L-proline, 0.1 mg/ml polysorbate 80, pH 5.5. In some embodiments, the anti-CD19 antibody (e.g., obexelimab) is formulated at a concentration of 80-200 mg/ml obexelimab, 1.5-3 mg/ml sodium acetate trihydrate, 0. 1-0.2 mg/ml acetic acid (at density 1.053 g/ml), 10-50 mg/ml L-proline, 0.05-0.2 mg/ml polysorbate 80, pH 5.0-6.0. In some embodiments, the anti-CD19 antibody (e.g., obexelimab) is formulated at a concentration of 122-127 mg/ml obexclimab, 2.0-2.5 mg/ml sodium acetate trihydrate, 0.15-0.19 mg/ml acetic acid (at density 1.053 g/ml), 25-35 mg/ml L-proline, 0.05-0.15 mg/ml polysorbate 80, pH 5.0-6.0.

In certain embodiments, formulation comprises the anti-CD19 antibody (e.g., obexelimab), one or more buffers, one or more tonicity modifiers, one or more solvents, and one or more surfactants. In some embodiments, the buffer can be a sodium acetate buffer. For example, the buffer can be sodium acetate trihydrate. In some embodiments, the tonicity modifier can be acetic acid, L-proline, and combinations thereof. In another embodiment, the solvent is water.

In some embodiments, the surfactant is a polysorbate. In some embodiments, the polysorbate is polysorbate-80. In some embodiments, a formulation comprises the anti-CD19 antibody (e.g., obexelimab), sodium acetate trihydrate, acetic acid and L-proline, water, and polysorbate-80.

In some embodiments, the formulation comprises the anti-CD19 antibody (e.g., obexclimab) in an amount from about 1 mg to about 500 mg per ml or about 50 mg to about 250 mg per ml or about 100 mg to about 250 mg per ml, sodium acetate trihydrate in an amount from about 1 to about 10 mg per ml or about 1 to about 5 mg per ml or about 1 to about 2.5 mg per ml, acetic acid and L-proline in an amount from about 5 to about 50 mg per ml or about 10 to about 50 mg per ml or about 20 to about 40 mg per ml, water up to about 1 ml, and polysorbate-80 in an amount from about 0.01 mg to about 1 mg per ml or about 0.01 to about 0.5 mg/ml or about 0.05 to about 0.2 mg/ml. Specifically, a formulation comprises the anti-CD19 antibody (e.g., obexelimab) in an amount from about 100 mg to about 250 mg/ml, sodium acetate trihydrate in an amount from about 1 to about 2.5 mg/ml, acetic acid and L-proline in an amount from about 20 to about 40 mg/ml, water up to about 1 mg/ml, and polysorbate-80 in an amount from about 0.05 to about 0.2 mg per ml.

The anti-CD19 antibodies (e.g., obexelimab) disclosed herein may also be formulated as immunoliposomes. A liposome is a small vesicle comprising various types of lipids, phospholipids and/or surfactant that is useful for delivery of an anti-CD19 antibody (e.g., obexclimab) to a mammal. Liposomes containing the anti-CD19 antibody (e.g., obexelimab) are prepared by methods known in the art. Liposomes with enhanced circulation time are disclosed in U.S. Pat. No. 5,013,556, incorporated entirely by reference. In some embodiments, the anti-CD19 antibody (e.g., obexelimab) is formulated in liposomes generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, the anti-CD19 antibody (e.g., obexclimab) is entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference.

In some embodiments, the anti-CD19 antibody (e.g., obexclimab) sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

In some embodiments, the liquid pharmaceutical composition comprises 122-127 mg/ml anti-CD19 antibody (e.g., obexclimab), 2.0-2.5 mg/ml sodium acetate trihydrate, at a pH 5.0-6.0, 0.15-0.19 mg/ml acetic acid (at density 1.053 g/ml), 25-35 mg/ml L-proline, 0.05-0.15 mg/ml polysorbate 80.

In some embodiments, the liquid pharmaceutical composition comprises about 125 mg/ml anti-CD19 antibody (e.g., obexelimab), 2.35 mg/ml sodium acetate trihydrate, 0.17 mg/ml acetic acid, 30 mg/ml L-proline, 0.1 mg/ml polysorbate 80 at pH 5.5.

In some embodiments, the liquid pharmaceutical composition comprises 122-127 mg/ml obexelimab, 2.0-2.5 mg/ml sodium acetate trihydrate, at a pH 5.0-6.0, 0.15-0.19 mg/ml acetic acid (at density 1.053 g/ml), 25-35 mg/ml L-proline, 0.05-0.15 mg/ml polysorbate 80.

In some embodiments, the liquid pharmaceutical composition comprises about 125 mg/ml obexelimab, 2.35 mg/ml sodium acetate trihydrate, 0.17 mg/ml acetic acid, 30 mg/ml L-proline, 0.1 mg/ml polysorbate 80 at pH 5.5.

### Enumerated Embodiments

Embodiment 1. A method of treating multiple sclerosis (MS) in a subject in need thereof comprising administering to the subject an anti-CD19 antibody comprising an Fc region modified by amino acid substitutions S267E and L328F, wherein the anti-CD19 antibody comprises a heavy chain HCDR1 comprising the amino acid sequence SYVMH (SEQ ID NO: 5), a HCDR2 comprising the amino acid sequence WIGYINPYNDGTKY (SEQ ID NO: 6), and a HCDR3 comprising an amino acid sequence GTYYYGTRVFDY (SEQ ID NO: 7), and the light chain LCDR1 comprising RSSKSLQNVNGNTYLY (SEQ ID NO: 2), a LCDR2 comprising the amino acid sequence RMSNLNS (SEQ ID NO: 3), and a LCDR3 comprising the amino acid sequence MQHLEYPIT (SEQ ID NO: 4) wherein administration of the anti-CD19 antibody improves, ameliorates or delays onset of one or more symptoms of MS.

Embodiment 2. A method of treating multiple sclerosis (MS) in a subject in need thereof comprising administering to the subject an anti-CD19 antibody comprising an Fc region modified by amino acid substitutions S267E and L328F, wherein the anti-CD19 antibody comprises a heavy chain variable region comprising an amino acid sequence that is at least 90% identical to SEQ ID NO: 12; and a light chain variable region that is at least 90% identical to comprising amino acid sequence SEQ ID NO: 11, wherein administration of the anti-CD19 antibody improves, ameliorates or delays onset of one or more symptoms of MS.

Embodiment 3. The method of embodiments 1 or 2, wherein the anti-CD19 antibody comprises a heavy chain variable region comprising amino acid sequence SEQ ID NO: 12; and a light chain variable region comprising amino acid sequence SEQ ID NO: 11.

Embodiment 4. The method of any one of the preceding embodiments, wherein the anti-CD19 antibody comprises: a heavy chain comprising an amino acid sequence of SEQ ID NO: 10; and a light chain comprising an amino acid sequence of SEQ ID NO: 9.

Embodiment 5. The method of any one of the preceding embodiments, wherein the anti-CD19 antibody is obexelimab.

Embodiment 6. The method of any one of the preceding embodiments, wherein the anti-CD19 antibody is administered intrathecally.

Embodiment 7. The method of any one of the preceding embodiments, wherein the anti-CD19 antibody is administered intravenously.

### EXAMPLES

### Example 1: Prevention of Induction of Disease Activity in EAE Model

Surrogate antibodies to obexelimab (Obx) and rituximab (anti-CD20) were evaluated in a MOG protein/pertussis EAE model. Briefly, huFcγRIIb transgenic mice (2B-KIX mice) were treated with a mouse surrogate of Obx (mObx), a mouse surrogate of rituximab, or a vehicle control (phosphate-buffered saline) twice per week. Mice treated with the Obx or rituximab surrogates received 10 mg/kg via intraperitoneal injection. One week following the administration of the initial treatment, mice were immunized with human MOG protein along with pertussis toxin delivered twice according to the standard protocol. Disease severity was assessed for individual mice daily for the following 27 days on a scale of 0 to 5, wherein an increasing clinical score corresponded with increased observed disease severity. Mice receiving a clinical score of 4 were euthanized. All mice in the vehicle control group were euthanized within 15 days of MOG injection. Two mice in the obexelimab surrogate group were euthanized on day 11 post MOG injection. One mouse in the rituximab surrogate group was euthanized on day 13 post MOG injection. Individual clinical scores of the mice are shown in the below Table 4.

TABLE 4
Observed Clinical Scores of Individual Mice in EAE Mouse Model.

| | Group | | | | | | | | | | | | | | | |
|---|---|---|---|---|---|---|---|---|---|---|---|---|---|---|---|---|
| Days | Vehicle Treated Mice | | | mObx (Obx Murine Surrogate) Treated Mice | | | | | | Rituximab Murine Surrogate Treated Mice | | | | | | |
| Post hMOG | Animal No. | | | Animal No. | | | | | | Animal No. | | | | | | |
| Injection | A1-1 | A1-3 | A2-2 | B1-1 | B1-2 | B1-3 | B1-4 | B2-1 | B2-2 | C1-1 | C1-2 | C1-3 | C1-4 | C2-1 | C-2-2 | C2-3 |
| 2 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 |
| 6 | 0 | 0 | 0 | 0 | 0 | 0 | 0 | 0 | 0 | 0 | 0 | 0 | 0 | 0 | 0 | 0 |
| 7 | 0 | 0 | 0 | 0 | 0 | 0 | 0 | 0 | 0 | 0 | 0 | 0 | 1 | 0 | 0 | 0 |
| 8 | 0 | 0 | 0 | 0 | 0 | 0 | 0 | 0 | 0 | 0 | 0 | 0 | 1 | 0 | 0 | 0 |
| 9 | 0 | 0 | 0 | 1 | 0 | 0 | 1 | 0 | 0 | 1 | 0 | 0 | 1 | 0 | 0 | 0 |
| 11 | 0 | 1 | 0 | 4* | 0 | 0 | 4* | 0 | 0 | 1 | 2 | 0 | 1 | 2 | 0 | 0 |
| 12 | 1 | 2 | 2 | | 0 | 0 | | 0 | 0 | 1 | 2 | 0 | 1 | 2 | 0 | 0 |
| 13 | 2 | 4* | 2 | | 0 | 0 | | 0 | 0 | 1 | 2 | 0 | 4* | 2 | 0 | 0 |
| 14 | 2 | | 4* | | 0 | 0 | | 0 | 0 | 1 | 2 | 0 | | 2 | 0 | 0 |
| 15 | 4* | | | | 0 | 0 | | 0 | 0 | 2 | 2 | 0 | | 2 | 0 | 0 |
| 16 | | | | | 0 | 0 | | 0 | 0 | 2 | 3 | 0 | | 2 | 0 | 0 |
| 17 | | | | | 0 | 0 | | 0 | 0 | 2 | 3 | 0 | | 2 | 0 | 0 |
| 18 | | | | | 0 | 0 | | 0 | 0 | 2 | 3 | 0 | | 2 | 0 | 0 |
| 20 | | | | | 0 | 0 | | 0 | 0 | 2 | 2 | 0 | | 2 | 0 | 0 |
| 21 | | | | | 0 | 0 | | 0 | 0 | 2 | 2 | 0 | | 2 | 0 | 0 |
| 23 | | | | | 0 | 0 | | 0 | 0 | 2 | 2 | 1 | | 2 | 1 | 0 |
| 26 | | | | | 0 | 0 | | 0 | 0 | 2 | 2 | 2 | | 2 | 3 | 0 |
| 27 | | | | | 0 | 0 | | 0 | 0 | 2 | 2 | 2 | | 2 | 4 | 0 |

*Animals were euthanized at a disease score of 4 due to severe EAE as per IACUC requirements.

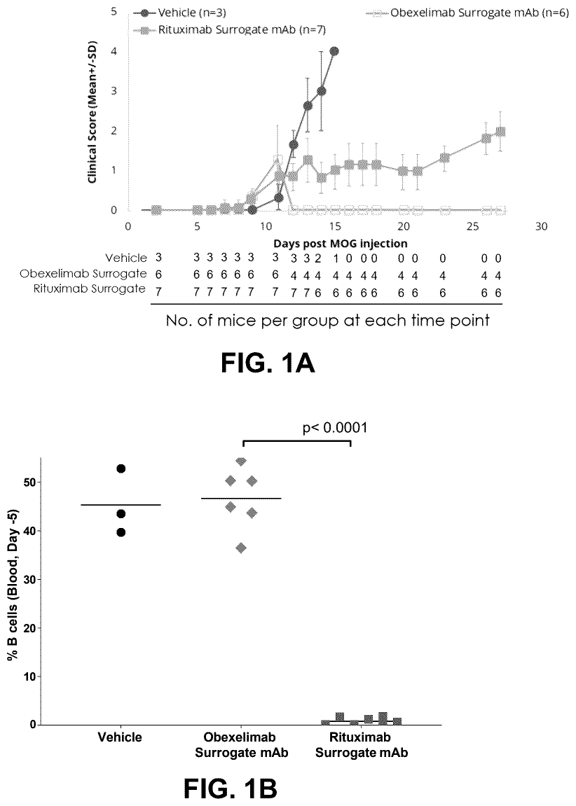

After 27 days, mObx showed prevention of disease as measured by standard clinical EAE scores of the surviving mice at each timepoint (FIG. 1A). Mice treated with mObx experienced a 33% (2/6) EAE incidence rate while mice treated with the rituximab surrogate experienced an 86% (6/7) EAE incidence rate and mice treated with the vehicle control experienced a 100% (3/3) EAE incidence rate. On Day-5, after initial treatment but prior to immunization of the mice with human MOG, a whole blood immune cell analysis was performed. Mice treated with mObx demonstrated a comparable blood B cell percentage as those mice treated with the vehicle control, while mice treated with the rituximab surrogate demonstrated a significant reduction in blood B cell percentages of total lymphocytes (FIG. 1B). The ability of mObx to suppress disease induction in the MOG-induced EAE model indicates the potential efficacy of Obx in preventing the onset of MS without depleting B cells in the blood.

### Example 2: A Murine Obexelimab Surrogate Reduces Mouse BCR-Mediated Phagocytosis

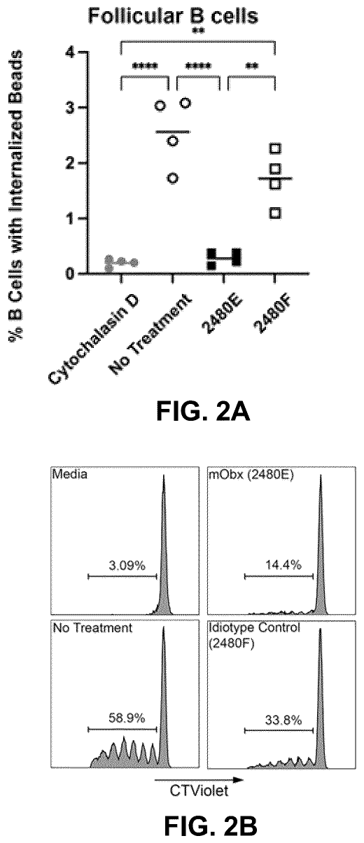

This example investigates if BCR-mediated phagocytosis is inhibited by 30 μg/ml mObx treatment. mObx was used to treat B cells isolated from 2B-KIX mice (Chu, S. Y., et al., 2021. J. Transl. Autoimmun. 4:100075) to understand how the different stages of B cell APC function and interactions with T cells are impacted in a standard immune response to foreign Ag. As shown in FIGS. 2A and 2B, mObx limits BCR-mediated Ag uptake and APC function to activate T cells ex vivo.

Spleen cells were isolated from 2B-KIX mice and treated for 1 hour with Cytochalasin D (an inhibitor of actin polymerization that blocks Ag uptake), media alone, mObx (2480E), or anti-CD19 that does not engage hFcγRIIb (2480F) as an idiotype control. After treatment, 2 μm fluorescent beads coated with anti-mouse IgM and biotinylated OVA were added to the media and cells were incubate for an additional 2 hours at 37° C. 5% CO₂. Bead uptake was analyzed by flow cytometry. Fo B cells were identified as CD19+ CD4-CD11b-IgMlo IgDhi CD23hi CD21int. Cells with internalized beads were identified as being Bead+, but not stained with fluorescent Streptavidin, indicating that the OVA-bio was internalized and not available for staining. Percent of total Fo B cells within internalized beads is shown (FIG. 2A). Each symbol represents cells isolated from an individual mouse (**p<0.01, ****p<0.0001).

B cells were isolated from lymphatic tissue harvested from 2B-KIX mice using negative selection beads and were then treated with media alone (Media and No Treatment), mObx (2480E), or idiotype control (2480F) for 1 hour 37° C. 5% CO₂. OVA-sp T cells were similarly harvested from the lymphatic tissue of OT-II mice and were labelled with CTViolet prior to being added to the B cell culture with no Ag (Media) or the anti-IgM/OVA-bio beads described above (all other groups). Cells were cocultured for 4 days and then T cell proliferation was analyzed by flow cytometry. Proliferated T cells were identified as CD4+ CD45R-cells with diluted CTViolet. One pilot example shown (FIG. 2B).

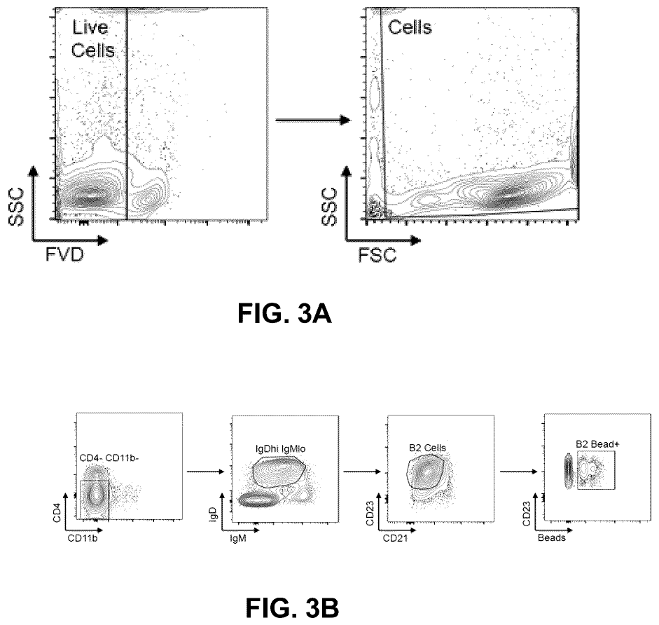

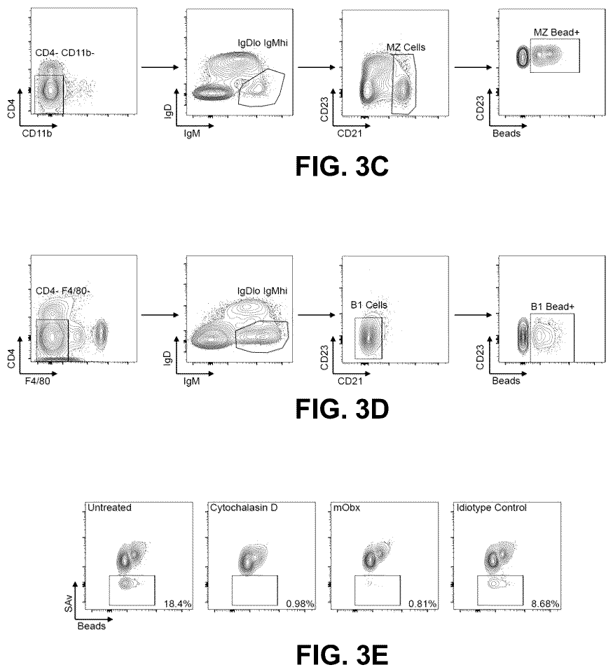

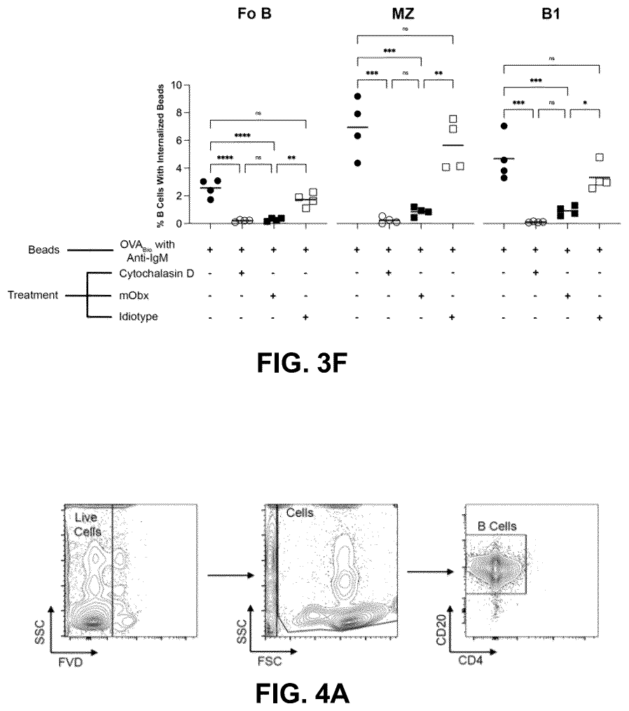

An ex vivo mouse B cell phagocytosis assay was also used. Splenic and peritoneal cavity cells were isolated from wildtype 2B-KIX mice and incubated with 2 μm beads and mObx for 2 hours, and B cells phagocytosis was subsequently assessed by flow cytometry (FIGS. 3A-D). Bead targets were coated with both anti-mouse IgM to target the BCR in an antigen-agnostic way and biotinylated OVA to allow for the use of a streptavidin stain to differentiate internalized bead from extracellular beads. Observing B cell phagocytosis, Fo B2, MZ, and B1 B cells were found to all be capable of phagocytosing anti-IgM coated beads. This capability was completely eliminated by treatment with cytochalasin D; a potent inhibitor of phagocytosis. Treatment with mObx at 30 μg/ml concentration significantly reduced phagocytic uptake of these beads compared to co-culture with the idiotype control (FIGS. 3E and 3F), demonstrating that mObx decreases mouse BCR-mediated antigen uptake conducted by B cells.

### Example 3: Obexelimab Inhibits Human BCR-Mediated Antigen Uptake and Phagocytosis

This example investigates if human BCR-mediated phagocytosis is inhibited by Obx treatment at 30 μg/ml using an ex vivo human B cell phagocytosis assay.

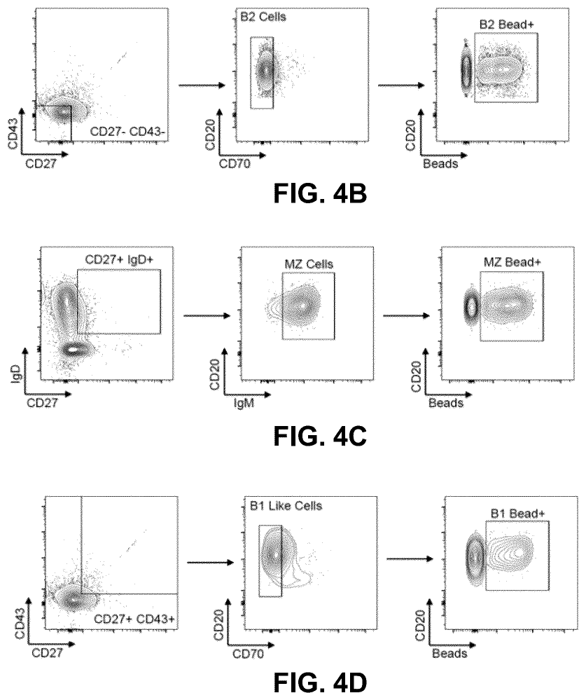

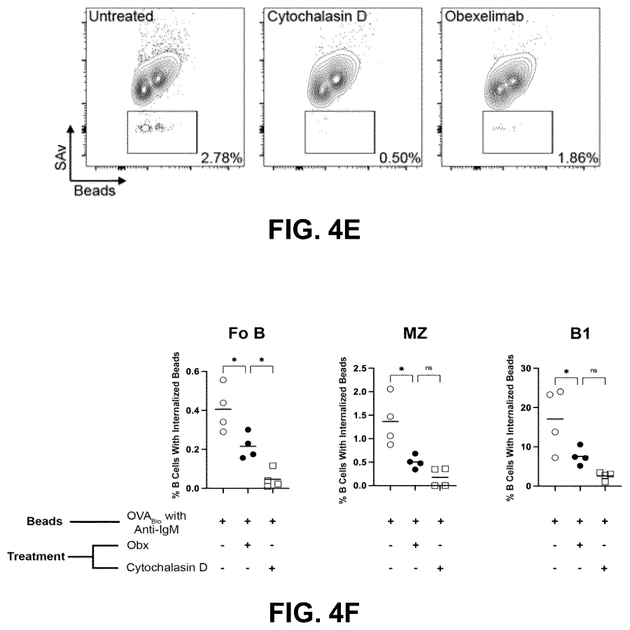

Human blood was drawn from healthy donors (n=4), and B cells were isolated and incubated with anti-human IgM conjugated beads and Obx for 2 hours, and B cell phagocytosis was subsequently assessed by flow cytometry (FIG. 4A-4D). Bead targets were coated with both anti-human IgM to target the BCR in an antigen-agnostic way and biotinylated OVA to allow for the use of a streptavidin stain to differentiate internalized bead from extracellular beads. Human Fo B2, MZ, and B1 B cells were all observed as capable of phagocytosing anti-human IgM coated beads. This capability was reduced by treatment with cytochalasin D, a potent inhibitor of phagocytosis. Treatment with Obx at 30 μg/ml concentration significantly reduced phagocytic uptake of these beads compared to the untreated control (FIGS. 4E and 4F), demonstrating that Obx decreases human BCR-mediated antigen uptake conducted by B cells.

### Example 4: B and T Cell Activation in Response to Antigen Stimulation is Reduced in the Presence of Obexelimab Surrogate (mObx)

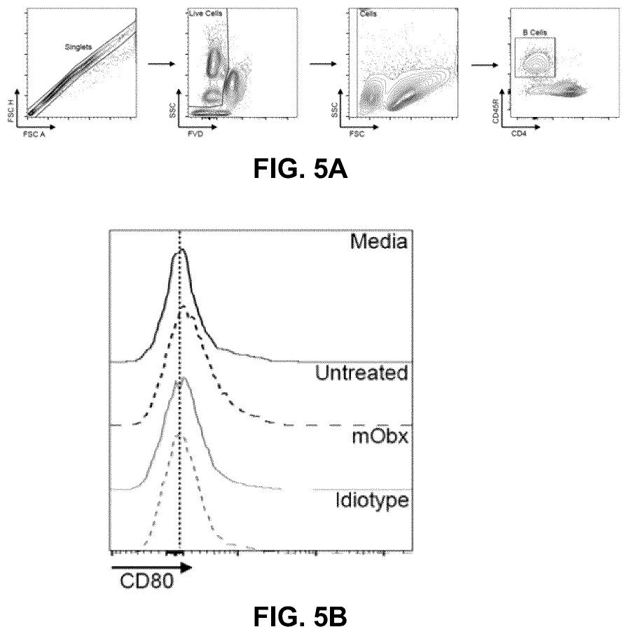

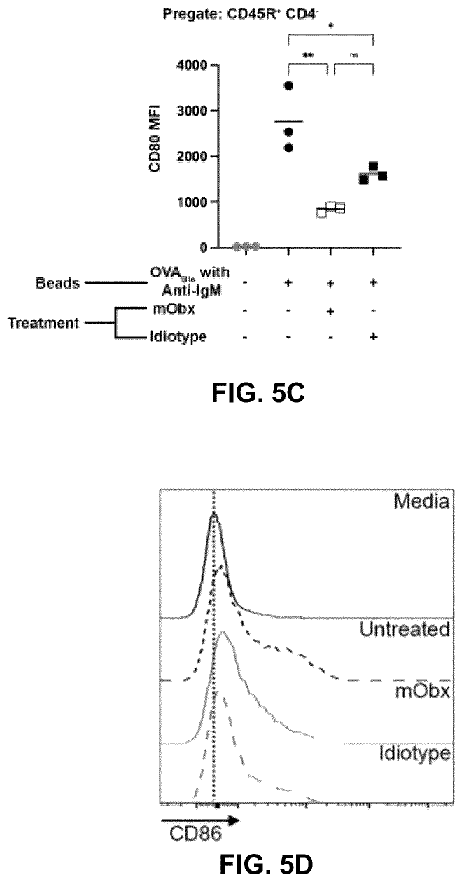

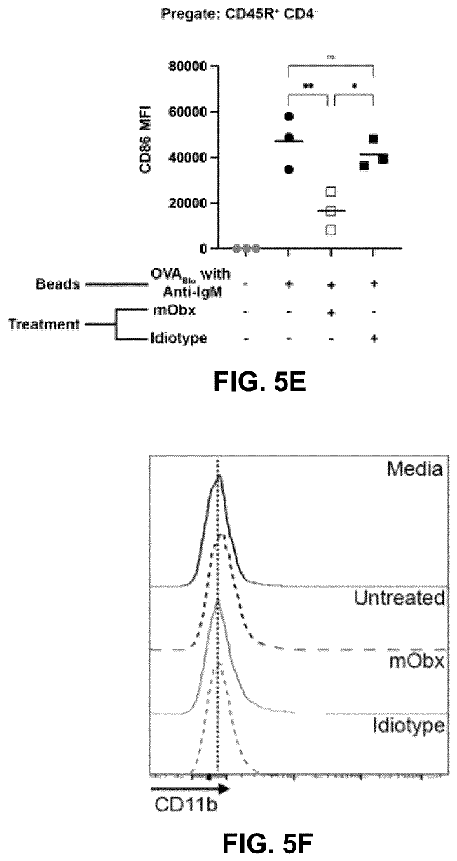

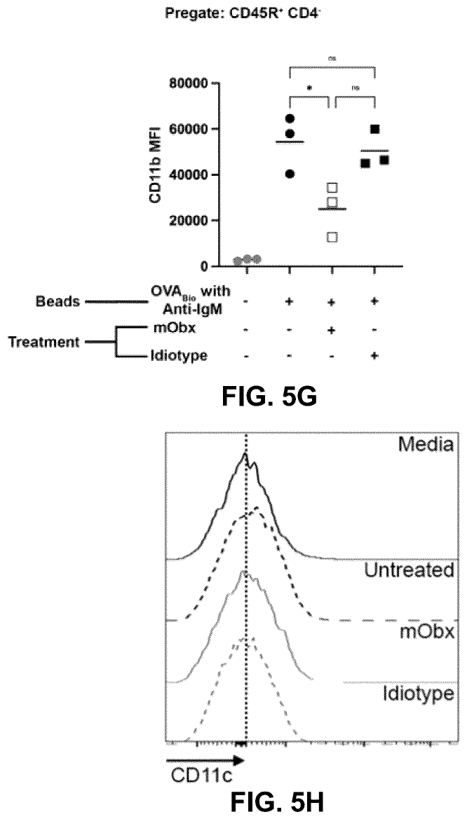

Following B cell antigen acquisition, B cells form direct interactions with T cells, where B cells act as the antigen presenting cell (APC) and present antigens that have been acquired to T cells resulting in B cell activation. To assess if treatment with mObx reduced B and T cell activation in response to B and T cell dependent interactions, B cells (5×10⁵) from 2B-KIX mice were pre-treated with mObx at 30 μg/ml for 1 hour prior to co-culture with CTViolet stained OT-II CD4 T cells (5×10⁵) and anti-mouse IgM biotinylated OVA conjugated beads (5×10⁵ beads) for 4 days. As T/B interactions are essential for B cell activation to T cell dependent antigens, B cell expression of activation markers using flow cytometry was analyzed (FIG. 5A). B cells co-cultured with bead bound antigen and antigen specific T cells had increased expression of CD80 and CD86 on their surface. mObx treatment significantly reduced this upregulation when compared to the untreated B cells (FIG. 5B-E). When compared to the idiotype control, B cells co-cultured with mObx expressed less CD80 and CD86 on their surface, demonstrating that B cells treated with mObx were less prone to co-stimulation and are thus, less activated. B cells co-cultured with bead bound antigen and antigen specific T cells had increased expression of CD11b and CD11e on their surface. Similarly, this increase was reduced if the B cells were also co-cultured with mObx when compared to the untreated B cells and the idiotype control treated B cells (FIG. 5F-I). Overall, these findings demonstrate that treatment with mObx at a concentration of 30 μg/ml can decrease T cell mediated B cell activation.

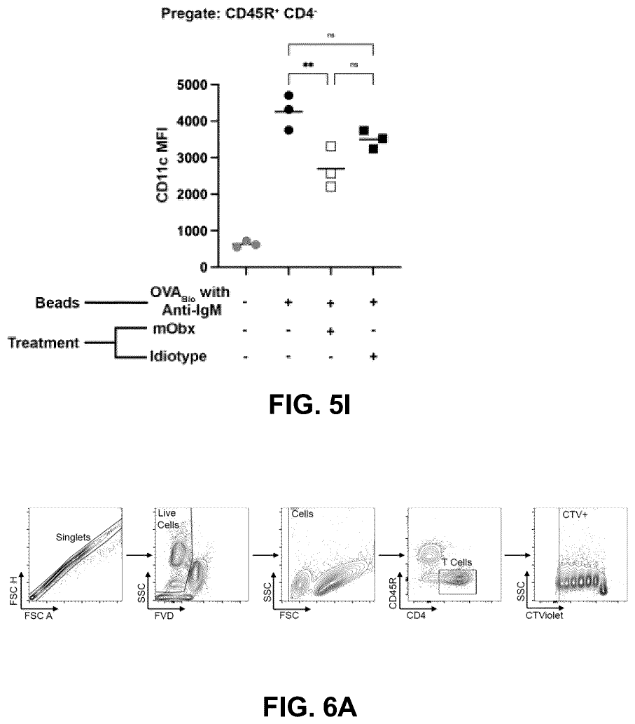

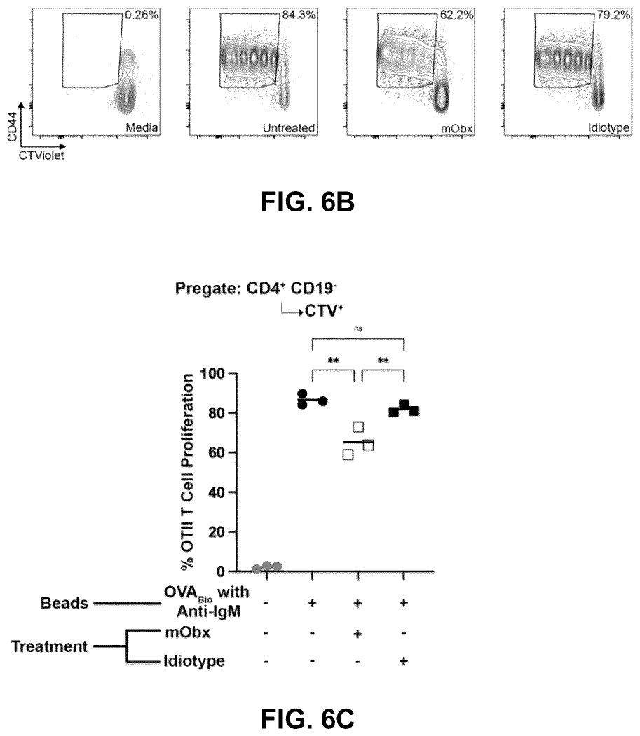

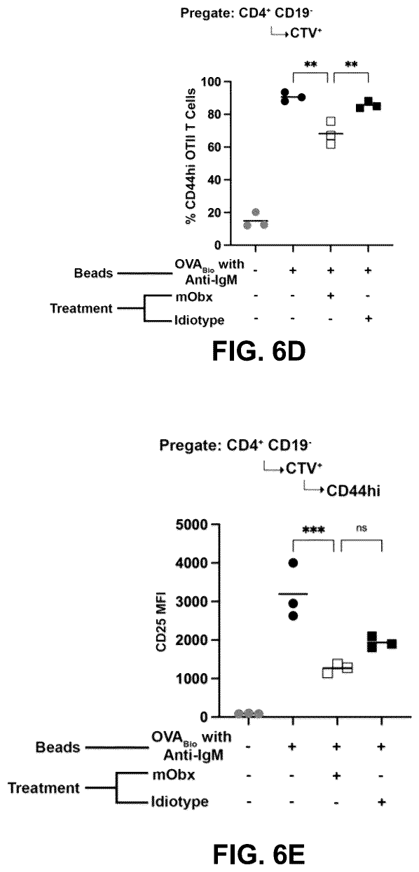

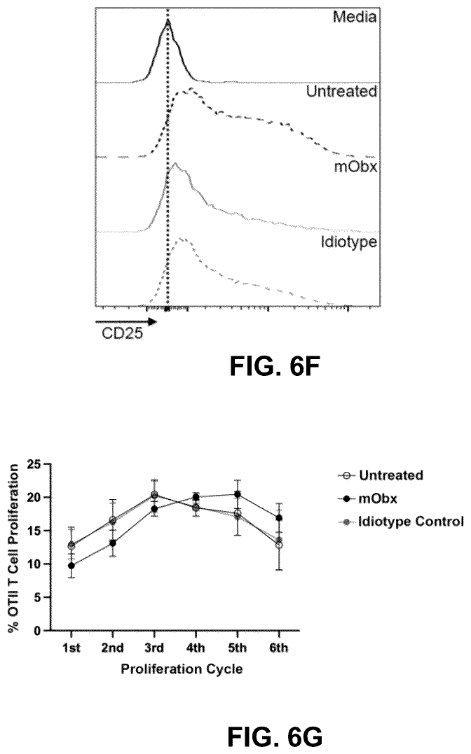

T cell activation markers were also evaluated using flow cytometry (FIG. 6A). It was observed that antigen specific CD4 T cells proliferated when co-cultured with antigen bound beads and B cells proliferated. However, CD4 T cells cultured with 30 μg/ml mObx treated B cells exhibited significantly diminished CD4 T cell proliferation when compared to co-culture with untreated B cells and B cells treated with the idiotype control (FIG. 6B, 6C). Additionally, CD4 T cells partnered with mObx treated B cells exhibited a significant reduction in surface level expression of CD44 when compared to co-culture with untreated and idiotype treated B cells (FIG. 6D), confirming reduced CD4 T cell activation and corroborating with reduced proliferation. When looking at the activated T Cells (CD4+ CD44+), it was observed that the activated T cells from co-culture with mObx treated B cells had a significant reduction in CD25 expression on their surface when compared to untreated B cells. CD25 expression was also reduced when compared to the idiotype control, although it was not significant (FIG. 6E, 6F). Therefore, the activation status of even proliferating CD4 T cells was lower in the presence of mObx. However, mObx-treated proliferating B cells underwent a similar number of divisions as untreated or idiotype-treated B cells (FIG. 6G), indicating that proliferation itself is not impacted. Together, this data suggests that B cells treated with mObx have a reduced ability to activate CD4 T cells.

### Example 5: Evaluation of B Cell Inhibition Vs Depletion as Approaches to Limit Systemic Vs Local B Cell Function

B cells are required for the initiation of several models of EAE. For example, B cells participate in initiating the autoimmune T cell response (likely as APCs) in the 2D2 IgH^{MOG} spontaneous version of EAE (SEAE) model where the number of MOG-sp B cells is highly elevated in lymphoid tissue (Dang, A. K., et al., Front. Immunol. 6:470, Bettelli et al., 2006, and Krishnamoorthy, G., H. et al., 2006. J. Clin. Invest. 116:2385-2392). Disease induced by human MOG (or humanized MOG, such as bMOGtag protein) in C57BL/6 mice depends on the generation of anti-MOG antibodies for initiation (Lyons, J.-A., et al., 2002. Eur. J. Immunol. 32:1905-1913, and Lu, Y., et al., 2022. J. Immunol. 209:2083-2092). Production of high-affinity antibodies depends on a germinal center response and, while this is MHC class II dependent, this is not equivalent to professional APC function to activate naïve T cells. The newly described PLPECD-induced model is unique in that B cell APC function (and not antibody formation) is required to both induce disease and sustain its progression (Boyden, A. W., et al., 2020. Sci. Rep. 10:5011, and Wilhelm, C. R., et al., 2023. J. Immunol. Ji2200721). Other induced models of EAE that incorporate both T and B cell recognition of antigen, such as mMOG_{tag}-induced EAE, do not depend on B cells for disease initiation, but MOG-sp B cells do appear to contribute to more severe disease (Dang, A. K et al., 2015. J. Neuroimmunol. 278C: 73-84).

Most current investigations of B cell APC function do not, or cannot, distinguish between potential B cell APC function that may occur in lymphoid tissue vs the inflamed meninges. It is difficult to target B cells in this location in large part because of the Blood-Brain/Meningeal Barriers. Indeed, a recent investigation of anti-CD20 treatment of sEAE mice showed that intravenous (i.v.) administered anti-CD20 rapidly bound B cells in lymphatic tissue, but was largely excluded from meningeal B cells, even in the presence of ongoing inflammation that is described to “disrupt” blood barriers (Tesfagiorgis, Y., E. et al., 2023. Systemic Administration of Anti-CD20 Indirectly Reduces B Cells in the Inflamed Meninges in a Chronic Model of Central Nervous System Autoimmunity). Lehmann-Horn et al. used intrathecal (i.t.) administration of anti-CD20 to try to directly target B cells in the CNS and meninges (Lehmann-Horn, K. S., et al., 2014. Ann. Clin. Transl. Neurol. 1:490-496). However, in these experiments peripheral B cells were also depleted to a similar degree as when anti-CD20 was administered i.v., indicating that i.t. administration is not restricted to the CNS.

This example evaluates obexelimab (Obx), a bi-specific monoclonal antibody that engages the inhibitory FcγRIIb specifically on CD19-expressing B cells to inhibit their function without resorting to destroying them (Chu, S. Y., et al., 2008. Mol. Immunol. 45:3926-3933) in a mouse model for MS. To determine if inhibition of B cell function in the periphery can prevent disease induction or progression, PLPECD-induced EAE mice (a model that has been shown to be uniquely dependent on B cells, but not antibodies, for disease progression (Boyden et al., 2020, and Wilhelm et al., 2023)) are administered either anti-CD20 or mObx i.v. 1 day prior to immunization with PLPECD or alternatively 14 days post immunization, corresponding with the onset of disease. Disease course is followed and mice are sacrificed once they reach the chronic phase of disease (2 weeks post onset). Blood and lymph nodes are analyzed by flow cytometry to quantify B cell numbers, confirm binding of anti-CD20 or mObx, as well as assay T cell activation and differentiation to effector subsets. Spinal cords are collected for immunofluorescence histology analysis of T and B cell inflammation in the meninges and white matter as well as demyelination, as described previously (Dang, A. K., et al., 2015. Front. Immunol. 6:470; Dang, A. K., et al., 2015. J. Neuroimmunol. 278C: 73-84, and Tesfagiorgis, Y., et al., 2017. J. Immunol. 199:449-457). Separate flow cytometry experiments of spinal cord cells are used to quantify T and B cell numbers in the tissue and to determine the extent of B cell binding by i.v.-administered anti-CD20 or mObx. This experiment is performed separately on male and female mice to determine if there is a sex difference in the therapeutic response to mObx.

### Example 6. Inhibition of Meningeal B Cell Function and Disease Progression

Intrathecal (i.t.) administration of monoclonal antibodies bypasses the Blood-Brain and Blood-Meningeal Barriers, but does not prevent leakage of i.t. administered antibodies to the periphery. Therefore, i.v. administration may be used to treat B cells outside of the CNS, while i.t. administration targets B cells both in the CNS/meninges and the periphery.

PLPECD-induced EAE mice are treated i.t. with anti-CD20 or mObx in the pre-symptomatic (1 day post immunization), acute (5 days post onset), or chronic phases (2 weeks post onset) of disease. Disease course is followed and, at the end of the experiment, spinal cords are harvested for analysis of local pathology and inflammation as described above. The organization of meningeal clusters, the ratio of T cells to B cells, and the physical interrelationship between these cells within the cluster tissue are evaluated, as it is possible that inhibition of B cells will change T:B interactions and localization. Flow cytometry is used to confirm that B cells are bound by i.t. administered drug and to quantify the number of infiltrating B cells and T cells as well as their activation status.

### Example 7. The Impact of B Cell Inhibition on T Cell: B Cell Interactions in the Meninges

B cells in the meninges may influence local inflammation through local APC function, presenting locally acquired self-antigen to reactivate newly recruited autoimmune T cells (Pipi et al., 2018). The actual presentation of Ag occurs through highly-coordinated, direct physical interactions between cells. This example evaluates the impact of mObx inhibition of B cell function on T:B interactions in lymph nodes or the meninges. Interactions between MOG-sp T and B cells using intravital microscopy early in the anti-MOG response in lymph nodes are directly visualized. MOG-specific B cells form a germinal center by presenting Ag to anti-MOG T cells in the early stages of the response following immunization with mMOGtag (Jain et al., 2018; Parham et al., 2022).

Activated MOG-sp RFP⁺ 2D2 T cells are transferred to either mMOGtag-induced or PLPECD-induced EAE in 2B-KIX mice in the acute phase of disease. At the same time, mice are treated i.t. with mObx or isotype control. Spinal cords are harvested for immunofluorescence histology 2 or 7 days post treatment to determine if RFP⁺ T cells are less associated with B cells in treated mice, if T cell numbers are reduced, or if fewer of these T cells are in the parenchyma or in the meninges.

Intravital microscopy of the inflamed spinal cord is used to directly visualize T cell interactions with meningeal B cells following mObx treatment as described above. Interaction duration between transferred RFP⁺ 2D2 T cells and endogenous B cells (B cell-specific conditional GFP mice (CD19cre×ROSA26-EGFP mice as recipients) is quantified. Further, imaging at the base of the meningeal clusters is performed to evaluate whether autoreactive T cells cross the border to the underlying white matter after interactions with B cells, following treatment with mObx.

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. A method of treating multiple sclerosis (MS) in a subject in need thereof comprising:
administering to the subject an anti-CD19 antibody comprising a light chain and a heavy chain,
wherein the light chain comprises a LCDR1 comprising an amino acid sequence of SEQ ID NO: 2, a LCDR2 comprising an amino acid sequence of SEQ ID NO: 3, and a LCDR3 comprising an amino acid sequence of SEQ ID NO: 4, and
wherein the heavy chain comprises a HCDR1 comprising an amino acid sequence of SEQ ID NO: 5, a HCDR2 comprising an amino acid sequence of SEQ ID NO: 6, and a HCDR3 comprising an amino acid sequence of SEQ ID NO: 7, and
wherein the heavy chain comprises an Fc region comprising amino acid substitutions 267E and L328F.

2. A method of treating multiple sclerosis (MS) in a subject in need thereof comprising:
administering to the subject an anti-CD19 antibody comprising a light chain and a heavy chain,
wherein the light chain comprises a light chain variable region that is at least 90% identical to an amino acid sequence of SEQ ID NO: 11, and
wherein the heavy chain comprises a heavy chain variable region that is at least 90% identical to an amino acid sequence of SEQ ID NO: 12, and
wherein the heavy chain comprises an Fc region comprising amino acid substitutions 267E and L328F.

3. The method of claim 1, wherein the anti-CD19 antibody comprises a heavy chain variable region comprising amino acid sequence SEQ ID NO: 12; and a light chain variable region comprising amino acid sequence SEQ ID NO: 11.

4. The method of claim 1, wherein the anti-CD19 antibody comprises a heavy chain and a light chain,
wherein the heavy chain comprises an amino acid sequence at least 90% identical to SEQ ID NO: 10; and
wherein the light chain comprises an amino acid sequence at least 90% identical to SEQ ID NO: 9.

5. The method of claim 1, wherein the anti-CD19 antibody comprises:
a heavy chain comprising an amino acid sequence of SEQ ID NO: 10; and
a light chain comprising an amino acid sequence of SEQ ID NO: 9.

6. The method of claim 1, wherein the anti-CD19 antibody is obexelimab.

7. The method of claim 1, wherein the administration of the anti-CD19 antibody improves, ameliorates, or delays the onset of one or more symptoms of MS.

8. The method of claim 1, wherein the anti-CD19 antibody is administered intrathecally.

9. The method of claim 1, wherein the anti-CD19 antibody is administered intravenously.

10. The method of claim 1, wherein the anti-CD19 antibody is administered subcutaneously.

11. The method of claim 1, wherein the anti-CD19 antibody is administered in a liquid formulation comprising 125 mg/ml anti-CD19 antibody, 2.35 mg/ml sodium acetate trihydrate, 0.17 mg/ml acetic acid, 30 mg/ml L-proline, 0.1 mg/ml polysorbate 80 at pH 5.5.

12. The method of claim 1, wherein the anti-CD19 antibody is administered using a prefilled syringe or autoinjector.


SEQUENCE LISTING (12 sequences)

SEQ ID NO: 1 - protein, 556 aa, Homo sapiens [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS